(19) United States
(12) Patent Application Publication
(10) Pub. No.: US 2006/0047114 A1
(43) Pub. Date: Mar. 2, 2006

(54) Azabicyclic amine histamine-3 receptor antagonists

(75) Inventor: Bishop Wlodecki, Preston, CT (US)

Correspondence Address: PFIZER INC, 150 EAST 42ND STREET, 5TH FLOOR - STOP 49, NEW YORK, NY, 10017-5612, US

(73) Assignee: Pfizer Inc

(21) Appl. No.: 11/212,268

(22) Filed: Aug. 26, 2005

Related U.S. Application Data
(60) Provisional application No. 60/606,688, filed on Sep. 1, 2004.
(60) Provisional application No. 60/647,874, filed on Jan. 27, 2005.

(51) Int. Cl. C07D 417/02; C07D 403/02; C07D 413/02

(52) U.S. Cl. 540/602; 544/106; 544/60; 544/143; 544/373; 546/200; 548/452

(57) ABSTRACT

This invention is directed to compounds of the formula I 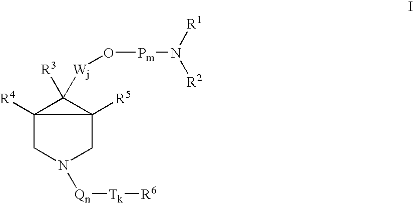 as defined herein, or a pharmaceutically acceptable salt thereof; a pharmaceutical composition containing a compound of formula I, a method of treatment of a disorder or condition that may be treated by antagonizing histamine H3 receptors, the method comprising administering to a mammal in need of such treatment a compound of formula I as described above, and a method of treatment of a disorder or condition selected from the group consisting of depression, mood disorders, schizophrenia, anxiety disorders, Alzheimer's disease, attention-deficit disorder (ADD), attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastrointestinal tract, the method comprising administering to a mammal in need of such treatment a compound of formula I as described above.

### BACKGROUND OF THE INVENTION

This invention is directed to compounds of formula I described herein, to a pharmaceutical composition comprising such compounds, and to methods of treatment of disorders or conditions that may be treated by antagonizing histamine-3 (H3) receptors using such compounds. The histamine-3 (H3) receptor antagonists of the invention are useful for treating anxiety disorders, including, for example, generalized anxiety disorder, panic disorder, PTSD, and social anxiety disorder; mood adjustment disorders, including depressed mood, mixed anxiety and depressed mood, disturbance of conduct, and mixed disturbance of conduct and depressed mood; age-associated learning and mental disorders, including Alzheimer's disease; attention adjustment disorders, such as attention-deficit disorders, or other cognitive disorders due to general medical conditions; attention-deficit hyperactivity disorder; psychotic disorders including schizoaffective disorders and schizophrenia; sleep disorders, including narcolepsy and enuresis; obesity; dizziness, epilepsy, and motion sickness. The H3 receptor antagonists of the invention are also useful for treating, for example, allergy, allergy-induced airway (e.g., upper airway) responses, congestion (e.g., nasal congestion), hypotension, cardiovascular disease, diseases of the GI tract, hyper- and hypo-motility and acidic secretion of the gastrointestinal tract, sleeping disorders (e.g., hypersomnia, somnolence, and narcolepsy), attention deficit hyperactivity disorder ADHD), hypo- and hyper-activity of the central nervous system (for example, agitation and depression), and other CNS disorders (such as schizophrenia and migraine).

Histamine is a well-known mediator in hypersensitive reactions (e.g. allergies, hay fever, and asthma) that are commonly treated with antagonists of histamine or “antihistamines.” It has also been established that histamine receptors exist in at least two distinct types, referred to as H1 and H2 receptors.

A third histamine receptor (H3 receptor) is believed to play a role in neurotransmission in the central nervous system, where the H3 receptor is thought to be disposed presynaptically on histaminergic nerve endings (Nature, 302, S32-837 (1983)). The existence of the H3 receptor has been confirmed by the development of selective H3 receptor agonists and antagonists (Nature, 327, 117-123 (1987)) and has subsequently been shown to regulate the release of the neurotransmitters in both the central nervous system and peripheral organs, particularly the lungs, cardiovascular system and gastrointestinal tract.

A number of diseases or conditions may be treated with histamine-3 receptor ligands wherein the H3 ligand may be an antagonist, agonist or partial agonist, see: (Imamura et al., Circulation Res., (1996) 78, 475-481); (Imamura et al., Circ. Res., (1996) 78, 863-869); (Lin et al., Brain Res. (1990) 523, 325-330); (Monti et al., Neuropsychopharmacology (1996) 15, 31-35); (Sakai et al., Life Sci. (1991) 48, 2397-2404); (Mazurkiewiez-Kwilecki and Nsonwah, Can. J. Physiol. Pharmacol. (1989) 67, 75-78); (Panula, P. et al., Neuroscience (1998) 44, 465-481); (Wada et al., Trends in Neuroscience (1991) 14, 415); (Monti et al., Eur. J. Pharmacol. (1991) 205, 283); (Haas et al., Behav. Brain Res. (1995) 66, 41-44); (De Almeida and Izquierdo, Arch. Int. Pharmacodyn. (1986) 283, 193-198); (Kamei et al., Psychopharmacology (1990) 102, 312-318); (Kamei and Sakata, Japan. J. Pharmacol. (1991) 57, 437-482); (Schwartz et al., Psychopharmacology; The Fourth Generation of Progress, Bloom and Kupfer (eds.), Raven Press, New York, (1995) 3, 97); (Shaywitz et al., Psychopharmacology (1984) 82, 73-77); (Dumery and Blozovski, Exp. Brain Res. (1987) 67, 61-69); (Tedford et al., J. Pharmacol. Exp. Ther. (1995) 275, 598-604); (Tedford et al., Soc. Neurosci. Abstr. (1996) 22, 22); (Yokoyama et al., Eur. J. Pharmacol. (1993) 234, 129); (Yokoyama and Iinuma, CNS Drugs (1996) 5, 321); (Onodera et al., Prog. Neurobiol. (1994) 42, 685); (Leurs and Timmerman, Prog. Drug Res. (1992) 39, 127); (The Histamine H3 Receptor, Leurs and Timmerman (ed.), Elsevier Science, Amsterdam, The Netherlands (1998); (Leurs et al., Trends in Pharm. Sci. (1998) 19, 177-183); (Phillips et al., Annual Reports in Medicinal Chemistry (1998) 33, 31-40); (Matsubara et al., Eur. J. Pharmacol. (1992) 224, 145); (Rouleau et al., J. Pharmacol. Exp. Ther. (1997) 281, 1085); (Adam Szelag, “Role of histamine H3-receptors in the proliferation of neoplastic cells in vitro”, Med. Sci. Monit., 4 (5): 747-755, (1998)); (Fitzsimons, C., H. Duran, F. Labombarda, B. Molinari and E. Rivera, “Histamine receptors signalling in epidermal tumor cell lines with H-ras gene alterations”, Inflammation Res., 47 (Suppl. 1): S50-S51, (1998)); (R. Leurs, R. C. Vollinga and H. Timmerman, “The medicinal chemistry and therapeutic potentials of ligand of the histamine H3 receptor”, Progress in Drug Research 45: 170-165, (1995)); (R. Levi and N. C. E. Smith, “Histamine H3-receptors: A new frontier in myocardial ischemia”, J. Pharm. Exp. Ther., 292: 825-830, (2000)); (Hatta, E., K Yasuda and R. Levi, “Activation of histamine H3 receptors inhibits carrier-mediated norepinephrine release in a human model of protracted myocardial ischemia”, J. Pharm. Exp. Ther., 283: 494-500, (1997); (H. Yokoyama and K. Iinuma, “Histamine and Seizures: Implications for the treatment of epilepsy”, CNS Drugs, 5 (5); 321-330, (1995)); (K. Hurukami, H. Yokoyama, K. Onodera, K. Iinuma and T. Watanabe, AQ-0 145, “A newly developed histamine H3 antagonist, decreased seizure susceptibility of electrically induced convulsions in mice”, Meth. Find. Exp. Clin. Pharmacol., 17 (C): 70-73, (1995); (Delaunois A., Gustin P., Garbarg M., and Ansay M., “Modulation of acetylcholine, capsaicin and substance P effects by histamine H3 receptors in isolated perfused rabbit lungs”, European Journal of Pharmacology 277 (2-3):243-50, (1995)); and (Dimitriadou, et al., “Functional relationship between mast cells and C-sensitive nerve fibres evidenced by histamine H3-receptor modulation in rat lung and spleen”, Clinical Science 87 (2):151-63, (1994). Such diseases or conditions include cardiovascular disorders such as acute myocardial infarction; memory processes, dementia and cognition disorders such as Alzheimer's disease and attention deficit hyperactivity disorder; neurological disorders such as Parkinson's disease, schizophrenia, depression, epilepsy, and seizures or convulsions; cancer such as cutaneous carcinoma,” medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

H3 receptor antagonists have also been previously described in, for example, WO 03/050099, WO 02/0769252, and WO 02/12224. The histamine H3 receptor (H3R) regulates the release of histamine and other neurotransmitters, including serotonin and acetylcholine. H3R is relatively neuron specific and inhibits the release of certain monoamines such as histamine. Selective antagonism of H3R raises brain histamine levels and inhibits such activities as food consumption while minimizing non-specific peripheral consequences. Antagonists of the receptor increase synthesis and release of cerebral histamine and other monoamines. By this mechanism, they induce a prolonged wakefulness, improved cognitive function, reduction in food intake and normalization of vestibular reflexes. Accordingly, the receptor is an important target for new therapeutics in Alzheimer's disease, mood and attention adjustments, including attention deficit hyperactive disorder (ADHD), cognitive deficiencies, obesity, dizziness, schizophrenia, epilepsy, sleeping disorders, narcolepsy and motion sickness, and various forms of anxiety.

The majority of histamine H3 receptor antagonists to date resemble histamine in possessing an imidazole ring that may be substituted, as described, for example, in WO 96/38142. Non-imidazole neuroactive compounds such as beta histamines (Arrang, Eur. J. Pharm. 1985, 111:72-84) demonstrated some histamine H3 receptor activity but with poor potency. EP 978512 and EP 982300 disclose non-imidazole alkylamines as histamine H3 receptor antagonists. WO 02/12224 (Ortho McNeil Pharmaceuticals) describes non-imidazole bicyclic derivatives as histamine H3 receptor ligands, and EP 1275647 (Les Laboratoires Servier) has disclosed novel octahydro-2H-pyrido[1,2-a]pyrazines that are selective H3 receptor antagonists. Other receptor antagonists have been described in WO 02/32893 and WO 02/06233.

This invention is directed to histamine-3 (H3) receptor antagonists of the invention useful for treating the conditions listed in the preceding paragraphs. The compounds of this invention are highly selective for the H3 receptor (vs. other histamine receptors), and possess remarkable drug disposition properties (pharmacokinetics). In particular, the compounds of this invention selectively distinguish H3R from the other receptor subtypes H1R, H2R. In view of the increased level of interest in histamine H3 receptor agonists, inverse agonists and antagonists in the art, novel compounds that interact with the histamine H3 receptor would be a highly desirable contribution to the art. The present invention provides such a contribution to the art being based on the finding that a novel class of azabicyclic compounds exhibits a high and specific affinity to the histamine H3 receptor.

### SUMMARY OF THE INVENTION

This invention is directed to compounds of the formula I: 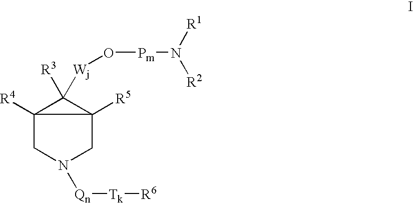
or the pharmaceutically acceptable salt(s) thereof, wherein:
P=methylene or a 3-8 member carbocyclic ring, optionally substituted by C₁-C₃ alkyl or fluorine;
T=methylene, optionally substituted by C₁-C₆ alkyl or cycloalkyl, OH, CN or phenyl (optionally substituted by Z as defined below);
Q=—(C═O)—, —SO₂—;
W═CR⁸R⁹;
j=0, 1 or 2;
k=0 to 6;
m=1 to 4;
n=0 or 1;
R¹ and R² are independently selected from the group that includes hydrogen, C₁-C₆ alkyl or C₃-C₇ cycloalkyl;
R¹ and R² together with the nitrogen to which they are attached form a 3-10 member cyclic or bicyclic ring, optionally with up to two additional heteroatoms selected from N, O, or S (e.g., azetidine, pyrrolidine, piperidine, homopiperidine, piperazine, morpholine, thiomorpholine);
R³, R⁴ and R⁵ are independently selected from the group consisting of hydrogen; C₁-C₄ alkyl, optionally substituted with 1 to 4 halogens (especially fluorine) or OH; C₃-C₇ cycloalkyl;
R⁶ is selected from the group that includes:
   aryl, optionally substituted with Z;
   heteroaryl, optionally substituted with Z;
R⁷ is selected from the list that includes:
   hydrogen;
   C₁-C₄ alkyl;
   C₃-C₇ cycloalkyl;
R⁸ and R⁹ are independently selected from the group that includes:
   hydrogen;
   C₁-C₄ alkyl;
   phenyl, optionally substituted with up to three of the atoms or functional groups defined for X below; or
R⁸ and R⁹ together with the carbon to which they are attached form a 3-7 member carbocyclic ring; and
X, Y and Z are independently selected from the group consisting of H, F, Cl, Br, I, CN, OH, NH₂, CF₃, C₂F₅, (C₁-C₆) alkyl, (C₁-C₆)-alkoxy or (C₁-C₆)alkyl-S(O)_{q}—, wherein q is 0, 1 or 2.

Where cis and trans isomers are possible for an embodiment of the inventive compound of formula I, both cis and trans isomers are within the scope of the invention.

The term “alkyl” refers to straight or branched chains of carbon atoms. Exemplary alkyl groups are C₁-C₆ alkyl groups which include methyl, ethyl, propyl, isopropyl, butyl, isobutyl, pentyl, isopentyl, hexyl, and the like, including all regioisomeric forms thereof, and straight and branched chain forms thereof. The term “alkyl” is also used to denote straight or branched chains of carbon atoms having one or more carbon-carbon double bonds, such as vinyl, allyl, butenyl, and the like, as well as straight or branched chains of carbon atoms having one or more carbon-carbon triple bonds, such as ethynyl, propargyl, butynyl, and the like. The term “aryl” denotes a cyclic, aromatic hydrocarbon. Examples of aryl groups include phenyl, naphthyl, anthracenyl, phenanthrenyl, and the like. The terms “alkoxy” and “aryloxy” denote “O-alkyl” and “O-aryl”, respectively. The term “cycloalkyl” denotes a cyclic group of carbon atoms, where the ring formed by the carbon atoms may be saturated or may comprise one or more carbon-carbon double bonds in the ring. Examples of cycloalkyl groups include cyclopropyl, cyclobutyl, cyclopentyl, cyclohexyl, cycloheptyl, and the like, as well as cyclopentenyl, cyclopentadienyl, cyclohexenyl, cyclohexadienyl, and the like. As used herein, the term “cycloalkyl” is also intended to denote a cyclic group comprising at least two fused rings, such as adamantanyl, decahydronaphthalinyl, norbornanyl, where the cyclic group may also have one or more carbon-carbon double bonds in one or both rings, such as in bicyclo[4.3.0]nona-3,6(1)-dienyl, dicyclopentadienyl, 1,2,3,4-tetrahydronaphthalinyl (tetralinyl), indenyl, and the like. The term “halogen” represents chloro, fluoro, bromo, and iodo. The term “heteroaryl” denotes a monocyclic or bicyclic aromatic group wherein one or more carbon atoms are replaced with heteroatoms selected from the group consisting of nitrogen, oxygen, and sulfur. If the heteroaryl group contains more than one heteroatom, the heteroatoms may be the same or different. Preferred heteroaryl groups are five- to fourteen-member rings that contain from one to three heteroatoms independently selected from oxygen, nitrogen, and sulfur. Examples of preferred heteroaryl groups include benzo[b]thienyl, chromenyl, furyl, imidazolyl, indazolyl, indolizinyl, indolyl, isobenzofuranyl, isoindolyl, isoquinolyl, isothiazolyl, isoxazolyl, naphthyridinyl, oxadiazolyl, oxazinyl, oxazolyl, phthalazinyl, pteridinyl, purinyl, pyranyl, pyrazinyl, pyrazolyl, pyridazinyl, pyridyl, pyrimidyl, pyrrolyl, quinolizinyl, quinolyl, quinoxalinyl, thiazolyl, thienyl, triazinyl, triazolyl, and xanthenyl.

The term “heterocycloalkyl” denotes a cycloalkyl system, wherein “cycloalkyl” is defined above, in which one or more of the ring carbon atoms are replaced with a heteroatom selected from the group consisting of nitrogen, oxygen, and sulfur. Examples of such heterocycloalkyl groups include azabicycloheptanyl, azetidinyl, benzazepinyl, 1,3-dihydroisoindolyl, indolinyl, tetrahydrofuryl, tetrahydroquinolinyl, tetrahydroisoquinolinyl, morpholinyl, piperazinyl, piperidyl, pyrrolidinyl, and, tetrahydro-2H-1,4-thiazinyl.

A cyclic group may be bonded to another group in more than one way. If no particular bonding arrangement is specified, then all possible arrangements are intended. For example, the term “pyridyl” includes 2-, 3-, or 4-pyridyl, and the term “thienyl” includes 2- or 3-thienyl.

The term “C₀-C₄” includes the embodiment where there are no carbons in a chain. Thus, for example, the groups “C₃-C₇ cycloalkyl-C₀-C₄ alkyl,” “C₆-C₁₄ aryl-C₀-C₄ alkyl,” “5-10-membered heteroaryl-C₀-C₄ alkyl,” and “C₆-C₁₄ aryl-C₀-C₄ alkylene-O—C₀-C₄ alkyl” include C₃-C₇ cycloalkyl, C₆-C₁₄ aryl, 5-10-membered heteroaryl, and C₆-C₁₄ aryl-O—C₀-C₄ alkyl, respectively.

The term “C₁-C₄ dialkylamino” refers to a dialkylamino group in which each alkyl group is independently a C₁-C₄alkyl group.

This invention is also directed to:
a pharmaceutical composition for treating, for example, a disorder or condition that may be treated by antagonizing histamine-3 receptors, the composition comprising a compound of formula I as described above, and optionally a pharmaceutically acceptable carrier;
a method of treatment of a disorder or condition that may be treated by antagonizing histamine-3 receptors, the method comprising administering to a mammal in need of such treatment a compound of formula I as described above; and
a pharmaceutical composition for treating, for example, a disorder or condition selected from the group consisting of depression, mood disorders, schizophrenia, anxiety disorders, Alzheimer's disease, attention deficit disorder (ADD), attention deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastrointestinal tract, the composition comprising a compound of formula I as described above, and optionally a pharmaceutically acceptable carrier.

This invention is also directed to a method of treatment of a disorder or condition selected from the group consisting of the disorders or conditions listed in the preceding paragraph, the method comprising administering to a mammal in need of such treatment a compound of formula I as described above.

The histamine-3 (H3) receptor antagonists of the invention are useful for treating, in particular, ADD, ADHD, obesity, anxiety disorders and respiratory diseases. Respiratory diseases that may be treated by the present invention include adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

The pharmaceutical composition and method of this invention may also be used for preventing a relapse in a disorder or condition described in the previous paragraphs. Preventing such relapse is accomplished by administering to a mammal in need of such prevention a compound of formula I as described above.

The disclosed compounds may also be used as part of a combination therapy, including their administration as separate entities or combined in a single delivery system, which employs an effective dose of a histamine H3 antagonist compound of general formula I and an effective dose of a histamine H1 antagonist, such as cetirizine (Zyrtec™), for the treatment of allergic rhinitis, nasal congestion and allergic congestion.

The disclosed compounds may also be used as part of a combination therapy, including their administration as separate entities or combined in a single delivery system, which employs an effective dose of a histamine H3 antagonist compound of general formula I and an effective dose of a neurotransmitter reuptake blocker. Examples of neurotransmitter reuptake blockers will include the serotonin-selective reuptake inhibitors (SSRI's) like sertraline (Zoloft™), fluoxetine (Prozac™), and paroxetine (Paxil™), or non-selective serotonin, dopamine or norepinephrine reuptake inhibitors for treating depression and mood disorders.

The compounds of the present invention may have optical centers and therefore may occur in different enantiomeric configurations. Formula I, as depicted above, includes all enantiomers, diastereomers, and other stereoisomers of the compounds depicted in structural formula I, as well as racemic and other mixtures thereof. Individual isomers can be obtained by known methods, such as optical resolution, optically selective reaction, or chromatographic separation in the preparation of the final product or its intermediate.

The present invention also includes isotopically labeled compounds, which are identical to those recited in formula I, but for the fact that one or more atoms are replaced by an atom having an atomic mass or mass number different from the atomic mass or mass number usually found in nature. Examples of isotopes that can be incorporated into compounds of the present invention include isotopes of hydrogen, carbon, nitrogen, oxygen, phosphorous, sulfur, fluorine and chlorine, such as ²H, ³H, ¹³C, ¹¹C, ¹⁴C, 15N, ¹⁸O, ¹⁷O, ³¹P, ³²P, ³⁵S, ¹⁸F, and ³⁶Cl, respectively. Compounds of the present invention, prodrugs thereof, and pharmaceutically acceptable salts of said compounds or of said prodrugs which contain the aforementioned isotopes and/or other isotopes of other atoms are within the scope of this invention. Certain isotopically labeled compounds of the present invention, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compounds of formula I of this invention and prodrugs thereof can generally be prepared by carrying out the procedures disclosed in the Schemes and/or in the Examples and Preparations below, by substituting a readily available isotopically labeled reagent for a non-isotopically labeled reagent.

“Antagonizing histamine-3 (H3) receptors,” as used herein, refers to acting as a histamine-3 receptor antagonist.

A “unit dosage form” as used herein is any form that contains a unit dose of the compound of formula I. A unit dosage form may be, for example, in the form of a tablet or a capsule. The unit dosage form may also be in liquid form, such as a solution or suspension.

The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation.

For oral administration, the pharmaceutical compositions may take the form of, for example, tablets or capsules prepared by conventional means with pharmaceutically acceptable excipients such as binding agents (e.g., pre-gelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., depression) is 0.1 to 200 mg of the active ingredient per unit dose which could be administered, for example, 1 to 4 times per day.

Aerosol formulations for treatment of the conditions referred to above (e.g., attention deficit hyperactivity disorder) in the average human are preferably arranged so that each metered dose or “puff” of aerosol contains 20 μg to 1000 μg of the compound of the invention. The overall daily dose with an aerosol will be within the range 100 μg to 100 mg. Administration may be several times daily, for example 2, 3, 4 or 8 times, giving for example, 1, 2 or 3 doses each time.

In connection with the use of an active compound of this invention with a histamine H1 antagonist, preferably cetirizine, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a histamine H1 antagonist, preferably cetirizine, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a histamine H1 antagonist) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a histamine H1 antagonist, preferably cetirizine, in the combination formulation for oral, parenteral or buccal administration to the average adult human for the treatment of the conditions referred to above is from about 0.1 mg to about 2000 mg, preferably from about 1 mg to about 200 mg of the histamine H1 antagonist per unit dose which could be administered, for example, 1 to 4 times per day.

A preferred dose ratio of cetirizine to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1>g to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a histamine H1 antagonist, preferably cetirizine, preferably from about 1 mg to about 200 mg of cetirizine. Administration may be several times daily, for example 2, 3, 4 or 8 times, giving for example, 1, 2 or 3 doses each time.

As previously indicated, a histamine H1 antagonist, preferably cetirizine, in combination with compounds of formula I are readily adapted to therapeutic use as antiallergy agents. In general, these antiallergy compositions containing a histamine H1 antagonist, preferably cetirizine, and a compound of formula I are normally administered in dosages ranging from about 0.01 mg to about 100 mg per kg of body weight per day of a histamine H1 antagonist, preferably cetirizine, preferably from about 0.1 mg. to about 10 mg per kg of body weight per day of cetirizine; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

In connection with the use of an active compound of this invention with a 5-HT re-uptake inhibitor, preferably sertraline, for the treatment of subjects possessing any of the above conditions, it is to be noted that these compounds may be administered either alone or in combination with pharmaceutically acceptable carriers by either of the routes previously indicated, and that such administration can be carried out in both single and multiple dosages. More particularly, the active combination can be administered in a wide variety of different dosage forms, i.e., they may be combined with various pharmaceutically-acceptable inert carriers in the form of tablets, capsules, lozenges, troches, hard candies, powders, sprays, aqueous suspension, injectable solutions, elixirs, syrups, and the like. Such carriers include solid diluents or fillers, sterile aqueous media and various non-toxic organic solvents, etc. Moreover, such oral pharmaceutical formulations can be suitably sweetened and/or flavored by means of various agents of the type commonly employed for such purposes. In general, the compounds of formula I are present in such dosage forms at concentration levels ranging from about 0.5% to about 95% by weight of the total composition, i.e., in amounts which are sufficient to provide the desired unit dosage and a 5-HT re-uptake inhibitor, preferably sertraline, is present in such dosage forms at concentration levels ranging from about 0.5% to about 95% by weight of the total composition, i.e., in amounts which are sufficient to provide the desired unit dosage.

A proposed daily dose of an active compound of this invention in the combination formulation (a formulation containing an active compound of this invention and a 5-HT re-uptake inhibitor) for oral, parenteral, rectal or buccal administration to the average adult human for the treatment of the conditions referred to above is from about 0.01 mg to about 2000 mg, preferably from about 0.1 mg to about 200 mg of the active ingredient of formula I per unit dose which could be administered, for example, 1 to 4 times per day.

A proposed daily dose of a 5-HT re-uptake inhibitor, preferably sertraline, in the combination formulation for oral, parenteral or buccal administration to the average adult human for the treatment of the conditions referred to above is from about 0.1 mg to about 2000 mg, preferably from about 1 mg to about 200 mg of the 5-HT re-uptake inhibitor per unit dose which could be administered, for example, 1 to 4 times per day.

A preferred dose ratio of sertraline to an active compound of this invention in the combination formulation for oral, parenteral or buccal administration to the average adult human for the treatment of the conditions referred to above is from about 0.00005 to about 20,000, preferably from about 0.25 to about 2,000.

Aerosol combination formulations for treatment of the conditions referred to above in the average adult human are preferably arranged so that each metered dose or “puff” of aerosol contains from about 0.01 μg to about 100 mg of the active compound of this invention, preferably from about 1 μg to about 10 mg of such compound. Administration may be several times daily, for example 2, 3, 4 or 8 times, giving for example, 1, 2 or 3 doses each time.

Aerosol formulations for treatment of the conditions referred to above in the average adult human are preferably arranged so that each metered dose or “puff” of aerosol contains from about 0.01 mg to about 2000 mg of a 5-HT re-uptake inhibitor, preferably sertraline, preferably from about 1 mg to about 200 mg of sertraline. Administration may be several times daily, for example 2, 3, 4 or 8 times, giving for example, 1, 2 or 3 doses each time.

As previously indicated, a 5-HT re-uptake inhibitor, preferably sertraline, in combination with compounds of formula I are readily adapted to therapeutic use as antidepressant agents. In general, these antidepressant compositions containing a 5-HT re-uptake inhibitor, preferably sertraline, and a compound of formula I are normally administered in dosages ranging from about 0.01 mg to about 100 mg per kg of body weight per day of a 5-HT re-uptake inhibitor, preferably sertraline, preferably from about 0.1 mg. to about 10 mg per kg of body weight per day of sertraline; with from about 0.001 mg. to about 100 mg per kg of body weight per day of a compound of formula I, preferably from about 0.01 mg to about 10 mg per kg of body weight per day of a compound of formula I, although variations will necessarily occur depending upon the conditions of the subject being treated and the particular route of administration chosen.

Anxiety disorders include, for example, generalized anxiety disorder, panic disorder, PTSD, and social anxiety disorder. Mood adjustment disorders include, for example, depressed mood, mixed anxiety and depressed mood, disturbance of conduct, and mixed disturbance of conduct and depressed mood. Attention adjustment disorders include, for example, in addition to ADHD, attention deficit disorders or other cognitive disorders due to general medical conditions. Psychotic disorders include, for example, schizoaffective disorders and schizophrenia; sleep disorders include, for example, narcolepsy and enuresis.

Examples of the disorders or conditions which may be treated by the compound, composition and method of this invention are also as follows: depression, including, for example, depression in cancer patients, depression in Parkinson's patients, post-myocardial Infarction depression, depression in patients with human immunodeficiency virus (HIV), Subsyndromal Symptomatic depression, depression in infertile women, pediatric depression, major depression, single episode depression, recurrent depression, child abuse induced depression, post partum depression, DSM-IV major depression, treatment-refractory major depression, severe depression, psychotic depression, post-stroke depression, neuropathic pain, manic depressive illness, including manic depressive illness with mixed episodes and manic depressive illness with depressive episodes, seasonal affective disorder, bipolar depression BP I, bipolar depression BP II, or major depression with dysthymia; dysthymia; phobias, including, for example, agoraphobia, social phobia or simple phobias; eating disorders, including, for example, anorexia nervosa or bulimia nervosa; chemical dependencies, including, for example, addictions to alcohol, cocaine, amphetamine and other psychostimulants, morphine, heroin and other opioid agonists, phenobarbital and other barbiturates, nicotine, diazepam, benzodiazepines and other psychoactive substances; Parkinson's diseases, including, for example, dementia in Parkinson's disease, neuroleptic-induced parkinsonism or tardive dyskinesias; headache, including, for example, headache associated with vascular disorders; withdrawal syndrome; age-associated learning and mental disorders; apathy; bipolar disorder; chronic fatigue syndrome; chronic or acute stress; conduct disorder; cyclothymic disorder; somatoform disorders such as somatization disorder, conversion disorder, pain disorder, hypochondriasis, body dysmorphic disorder, undifferentiated disorder, and somatoform NOS; incontinence; inhalation disorders; intoxication disorders; mania; oppositional defiant disorder; peripheral neuropathy; post-traumatic stress disorder; late luteal phase dysphoric disorder; specific developmental disorders; SSRI “poop out” syndrome, or a patient's failure to maintain a satisfactory response to SSRI therapy after an initial period of satisfactory response; and tic disorders including Tourette's disease.

As an example, the mammal in need of the treatment or prevention may be a human. As another example, the mammal in need of the treatment or prevention may be a mammal other than a human.

A compound of formula I, which is basic in nature, is capable of forming a wide variety of different salts with various inorganic and organic acids. The acid addition salts are readily prepared by treating the base compounds with a substantially equivalent amount of the chosen mineral or organic acid in an aqueous solvent medium or in a suitable organic solvent such as methanol or ethanol. Upon careful evaporation of the solvent, the desired solid salt is obtained.

The acids which are used to prepare the pharmaceutically acceptable acid salts of the active compound used in formulating the pharmaceutical composition of this invention that are basic in nature are those which form non-toxic acid addition salts, i.e., salts containing pharmacologically acceptable anions. Non-limiting examples of the salts include the acetate, benzoate, beta-hydroxybutyrate, bisulfate, bisulfite, bromide, butyne-1,4-dioate, caproate, chloride, chlorobenzoate, citrate, dihydrogenphosphate, dinitrobenzoate, fumarate, glycollate, heptanoate, hexyne-1,6-dioate, hydroxybenzoate, iodide, lactate, maleate, malonate, mandelate, metaphosphate, methanesulfonate, methoxybenzoate, methylbenzoate, monohydrogen phosphate, naphthalene-1-sulfonate, naphthalene-2-sulfonate, oxalate, phenylbutyrate, phenylpropionate, phosphate, phthalate, phenylacetate, propanesulfonate, propiolate, propionate, pyrophosphate, pyrosulfate, sebacate, suberate, succinate, sulfate, sulfite, sulfonate, tartrate, xylenesulfonate, acid phosphate, acid citrate, bitartrate, succinate, gluconate, saccharate, nitrate, methanesulfonate and pamoate [i.e., 1,1′-methylene-bis-(2-hydroxy-3-naphthoate)] salts.

Preferred embodiments of the present invention include the compounds of formula I in which
(A) R¹ and R² together with the nitrogen to which they are attached form a piperidine ring; or
(B) R¹ and R² together with the nitrogen to which they are attached form a pyrrolidine ring; or
(C) R¹ and R² are each independently methyl.

The most preferred embodiment of the present invention include the compounds of formula I in which R¹ and R² together with the nitrogen to which they are attached form a piperidine ring, R³, R⁴ and R⁵ are hydrogen, R⁶ is phenyl, k=0, m=3 and n=1.

Preferred embodiments of the present invention also include any combination of the foregoing embodiments (A)-(C).

Preferred compounds of formula I in accordance with the present invention are the following:
3-(3,4-dichlorobenzyl)-6-(3-piperidin-1-ylpropoxymethyl)-3-azabicyclo[3.1.0]hexane;
3-(4-chlorobenzyl)-6-(3-morpholin-4-ylpropoxymethyl)-3-azabicyclo[3.1.0]hexane;
3-(4-methoxybenzyl)-6-(3-thiomorpholin-4-ylpropoxymethyl)-3-azabicyclo[3.1.0]-hexane;
N-isopropyl-N-methyl-[3-(3-pyridin-2-ylmethyl-3-azabicyclo[3.1.0]hex-6-ylmethoxy)-propyl]-amine;
[2-(3-pyrimidin-2-ylmethyl-3-azabicyclo[3.1.0]hex-6-ylmethoxy)-ethyl]-dicyclopropyl-amine;
{6-[2-(2,4-dimethylazetidin-1-yl)-ethoxymethyl]-3-azabicyclo[3.1.0]hex-3-yl}-pyridin-4-ylmethanone;
6-(2-pyrrolidin-1-ylethoxymethyl)-3-azabicyclo[3.1.0]hexane-3-carboxylic acid methyl-(3-trifluoromethylphenyl)-amide;
1,5-dimethyl-3-phenethyl-6-(2-pyrrolidin-1-ylethoxymethyl)-3-azabicyclo[3.1.0]hexane;
[2-(3-benzenesulfonyl-3-azabicyclo[3.1.0]hex-6-ylmethoxy)-ethyl]-diethylamine;
{2-[3-(1H-indole-6-sulfonyl)-3-azabicyclo[3.1.0]hex-6-ylmethoxy]-ethyl}-dimethylamine;
[6-(2-dimethylamino-ethoxymethyl)-6-methyl-3-azabicyclo[3.1.0]hex-3-yl]-phenyl-methanone;
6-(2-pyrrolidin-1-ylethoxymethyl)-3-azabicyclo[3.1.0]hexane-3-carboxylic acid phenyl-amide;
6-(3-pyrrolidin-1-ylpropoxymethyl)-3-azabicyclo[3.1.0]hexane-3-carboxylic acid p-tolylamide;
1-{4-[2-(3-benzyl-3-azabicyclo[3.1.0]hex-6-ylmethoxy)-ethyl]-piperazin-1-yl}-ethanone;
3-benzyl-6-[2-(4-methanesulfinyl-piperazin-1-yl)-ethoxymethyl]-3-azabicyclo[3.1.0]-hexane;
3-benzyl-6-{2-[4-(propane-2-sulfonyl)-piperazin-1-yl]-ethoxymethyl}-3-azabicyclo[3.1.0]hexane;
3-benzyl-6-{2-[4-(4-chlorobenzenesulfonyl)-piperazin-1-yl]-ethoxymethyl}-3-azabicyclo[3.1.0]hexane;
3-benzyl-6-{2-[4-(3-fluorophenyl)-piperazin-1-yl]-ethoxymethyl}-3-azabicyclo[3.1.0]-hexane;
3-benzyl-6-[2-(4-pyridin-2-ylpiperazin-1-yl)-ethoxymethyl]-3-azabicyclo[3.1.0]hexane;
3-(2-methylbenzyl)-6-[2-(4-pyrimidin-2-ylpiperazin-1-yl)-ethoxymethyl]-3-azabicyclo-[3.1.0]hexane;
6-[2-(2,5-dimethylpyrrolidin-1-yl)-ethoxymethyl]-3-(2-methoxybenzyl)-3-azabicyclo-[3.1.0]hexane;
{2-[3-(1-phenylethyl)-3-azabicyclo[3.1.0]hex-6-ylmethoxy]-ethyl}-dimethylamine;
3-benzyl-6-[3-(3,5-dimethyl morpholin-4-yl)-propoxymethyl]-3-azabicyclo[3.1.0]-hexane;
3-benzyl-6-(1-methyl-2-pyrrolidin-1-ylethoxymethyl)-3-azabicyclo[3.1.0]hexane;
[6-(2-pyrrolidin-1-ylpropoxymethyl)-3-azabicyclo[3.1.0]hex-3-yl]-phenyl-methanone;
[6-(2-dimethylamino-ethoxymethyl)-3-azabicyclo[3.1.0]hex-3-yl]-(6-fluoronaphthalen-2-yl)-methanone;
[6-(2-dimethylamino-propoxymethyl)-3-azabicyclo[3.1.0]hex-3-yl]-naphthalen-2-yl-methanone;
[6-(2-dimethylamino-propoxymethyl)-3-azabicyclo[3.1.0]hex-3-yl]-quinolin-3-yl-methanone;
[6-(2-pyrrolidin-1-ylpropoxymethyl)-3-azabicyclo[3.1.0]hex-3-yl]-quinolin-3-yl-methanone;
3-benzyl-6-(4-piperidin-1-ylbutoxymethyl)-3-azabicyclo[3.1.0]hexane;
(4-methoxyphenyl)-[6-(4-piperidin-1-ylbutoxymethyl)-3-azabicyclo[3.1.0]hex-3-yl]-methanone;
3-(3-chlorobenzenesulfonyl)-6-(4-pyrrolidin-1-ylbutoxymethyl)-3-azabicyclo[3.1.0]-hexane;
3-benzenesulfonyl-1,5-dimethyl-6-(4-pyrrolidin-1-ylbutoxymethyl)-3-azabicyclo[3.1.0]-hexane;
3-benzyl-6-(4-piperidin-1-ylcyclohexyloxymethyl)-3-azabicyclo[3.1.0]hexane;
[3-(3-benzyl-3-azabicyclo[3.1.0]hex-6-ylmethoxy)-cyclopentyl]-dimethylamine;
3-benzyl-6-(3-morpholin-4-ylcyclobutoxymethyl)-3-azabicyclo[3.1.0]hexane;
3-(4-chlorobenzyl)-6-(2-pyrrolidin-1-ylcyclopropoxymethyl)-3-azabicyclo[3.1.0]hexane;
3-benzyl-6-(3-pyrrolidin-1-ylbicyclo[3.2.1]oct-8-yloxymethyl)-3-azabicyclo[3.1.0]-hexane; and
[5-(3-benzyl-3-azabicyclo[3.1.0]hex-6-ylmethoxy)-octahydro-pentalen-2-yl]-dimethylamine.

The most preferred examples of compounds according to the present invention include:
(1S,5R,6R)-3-benzyl-6-(3-piperidin-1-ylpropoxymethyl)-3-azabicyclo[3.1.0]hexane;
(1S,5R,6R)-1-{4-[6-(3-piperidin-1-ylpropoxymethyl)-3-azabicyclo[3.1.0]hex-3-ylmethyl]-phenyl}-ethanone;
(1S,5R,6R)-3-naphthalen-2-ylmethyl-6-(3-piperidin-1-ylpropoxymethyl)-3-azabicyclo[3.1.0]hexane;
(1S,5R,6R)-6-(3-piperidin-1-ylpropoxymethyl)-3-pyridin-3-ylmethyl-3-azabicyclo[3.1.0]hexane;
(1S,5R,6R)-3-(4-methoxybenzyl)-6-(3-piperidin-1-ylpropoxymethyl)-3-azabicyclo[3.1.0]hexane;
(1S,5R,6R)-3-(4-methylbenzyl)-6-(3-piperidin-1-ylpropoxymethyl)-3-azabicyclo[3.1.0]hexane;
(1S,5R,6R)-3-(3-fluorobenzyl)-6-(3-piperidin-1-ylpropoxymethyl)-3-azabicyclo[3.1.0]hexane;
3-[(1S,5R,6R)-6-(3-piperidin-1-ylpropoxymethyl)-3-azabicyclo[3.1.0]hex-3-ylmethyl]-phenol;
4-[(1S,5R,6R)-6-(3-piperidin-1-ylpropoxymethyl)-3-azabicyclo[3.1.0]hex-3-ylmethyl]-phenol;
4-[(1S,5R,6R)-6-(3-piperidin-1-ylpropoxymethyl)-3-azabicyclo[3.1.0]hex-3-ylmethyl]-benzonitrile;
(1S,5R,6R)-3-(3-phenoxybenzyl)-6-(3-piperidin-1-ylpropoxymethyl)-3-azabicyclo[3.1.0]hexane;
(1S,5R,6R)-3-(3-benzyloxybenzyl)-6-(3-piperidin-1-ylpropoxymethyl)-3-azabicyclo[3.1.0]hexane;
(1S,5R,6R)-3-(4-butoxybenzyl)-6-(3-piperidin-1-ylpropoxymethyl)-3-azabicyclo[3.1.0]hexane;
(1S,5R,6R)-3-biphenyl-4-ylmethyl-6-(3-piperidin-1-ylpropoxymethyl)-3-azabicyclo[3.1.0]hexane;
(1S,5R,6R)-3-benzo[1,3]dioxol-5-ylmethyl-6-(3-piperidin-1-ylpropoxymethyl)-3-azabicyclo[3.1.0]hexane;
(1S,5R,6R)-6-(3-piperidin-1-ylpropoxymethyl)-3-(3-trifluoromethylbenzyl)-3-azabicyclo[3.1.0]hexane;
(1S,5R,6R)-3-(4-bromobenzyl)-6-(3-piperidin-1-ylpropoxymethyl)-3-azabicyclo[3.1.0]hexane;
(1S,5R,6R)-3-(4-isopropylbenzyl)-6-(3-piperidin-1-ylpropoxymethyl)-3-azabicyclo[3.1.0]hexane;
(1S,5R,6R)-3-(3-chlorobenzyl)-6-(3-piperidin-1-ylpropoxymethyl)-3-azabicyclo[3.1.0]hexane;
(1S,5R,6R)-3-(2,3-dihydro-benzo[1,4]dioxin-6-ylmethyl)-6-(3-piperidin-1-ylpropoxymethyl)-3-azabicyclo[3.1.0]hexane;
(1S,5R,6R)-3-(4-ethoxybenzyl)-6-(3-piperidin-1-ylpropoxymethyl)-3-azabicyclo[3.1.0]hexane;
(1S,5R,6R)-3-(4-tert-butylbenzyl)-6-(3-piperidin-1-ylpropoxymethyl)-3-azabicyclo[3.1.0]hexane;
3-[(1S,5R,6R)-6-(3-piperidin-1-ylpropoxymethyl)-3-azabicyclo[3.1.0]hex-3-ylmethyl]-benzonitrile;
(1S,5R,6R)-3-(3,5-dichlorobenzyl)-6-(3-piperidin-1-ylpropoxymethyl)-3-azabicyclo[3.1.0]hexane;
(1S,5R,6R)-3-benzo[1,3]dioxol-4-ylmethyl-6-(3-piperidin-1-ylpropoxymethyl)-3-azabicyclo[3.1.0]hexane;
(1S,5R,6R)-6-(3-piperidin-1-ylpropoxymethyl)-3-(4-trifluoromethylbenzyl)-3-azabicyclo[3.1.0]hexane;
(1S,5R,6R)-3-(4-phenoxybenzyl)-6-(3-piperidin-1-ylpropoxymethyl)-3-azabicyclo[3.1.0]hexane;
(1S,5R,6R)-3-(2,6-difluorobenzyl)-6-(3-piperidin-1-ylpropoxymethyl)-3-azabicyclo[3.1.0]hexane;
(1S,5R,6R)-3-(4-methylsulfanylbenzyl)-6-(3-piperidin-1-ylpropoxymethyl)-3-azabicyclo[3.1.0]hexane;
(1R,5S,6R)-3-(3,5-difluorobenzyl)-6-[(3-piperidin-1-ylpropyl)oxymethyl]-3-azabicyclo[3.1.0]hexane;
(1R,5S,6R)-6-(3-piperidin-1-ylpropoxymethyl)-3-(4-propoxybenzyl)-3-azabicyclo[3.1.0]hexane;
(1R,5S,6R)-3-(4-fluoro-3-trifluoromethylbenzyl)-6-[(3-piperidin-1-ylpropyl)oxymethyl]-3-azabicyclo[3.1.0]hexane;
(1R,5S,6R)-3-(4-tert-butoxybenzyl)-6-(3-piperidin-1-ylpropoxymethyl)-3-azabicyclo[3.1.0]hexane;
5-[(1R,5S,6R)-6-(3-piperidin-1-ylpropoxymethyl)-3-azabicyclo[3.1.0]hex-3-ylmethyl]-benzene-1,3-diol;
(1R,5S,6R)-6-(3-piperidin-1-ylpropoxymethyl)-3-(4-trifluoromethoxybenzyl)-3-azabicyclo[3.1.0]hexane;
(1R,5S,6R)-3-(3-ethoxy-4-methoxybenzyl)-6-[(3-piperidin-1-ylpropyl)oxymethyl]-3-azabicyclo[3.1.0]hexane;
(1R,5S,6R)-6-(3-piperidin-1-ylpropoxymethyl)-3-(4-trifluoromethylsulfanylbenzyl)-3-azabicyclo[3.1.0]hexane;
(1R,5S,6R)-3-(4-ethylbenzyl)-6-(3-piperidin-1-ylpropoxymethyl)-3-azabicyclo[3.1.0]hexane;
(1R,5S,6R)-3-(4-isopropoxybenzyl)-6-(3-piperidin-1-ylpropoxymethyl)-3-azabicyclo[3.1.0]hexane;
(1R,5S,6R)-3-(3,5-dimethylbenzyl)-6-[(3-piperidin-1-ylpropyl)oxymethyl]-3-azabicyclo[3.1.0]hexane;
(1R,5S,6R)-3-(2′-methylbiphenyl-4-ylmethyl)-6-(3-piperidin-1-ylpropoxymethyl)-3-azabicyclo[3.1.0]hexane;
2-{4-[(1R,5S,6R)-6-(3-piperidin-1-ylpropoxymethyl)-3-azabicyclo[3.1.0]hex-3-ylmethyl]-phenoxy}-ethanol;
(1R,5S,6R)-3-(4-isobutylbenzyl)-6-(3-piperidin-1-ylpropoxymethyl)-3-azabicyclo[3.1.0]hexane;
(1R,5S,6R)-3-[4-(4-fluorophenoxy)-benzyl]-6-(3-piperidin-1-ylpropoxymethyl)-3-azabicyclo[3.1.0]hexane;
(1R,5S,6R)-3-(2,2-dimethylchroman-6-ylmethyl)-6-[(3-piperidin-1-ylpropyl)oxymethyl]-3-azabicyclo[3.1.0]hexane;
N-{4-[(1R,5S,6R)-6-(3-piperidin-1-ylpropoxymethyl)-3-azabicyclo[3.1.0]hex-3-ylmethyl]-phenyl}-acetamide;
6-[(1R,5S,6R)-6-(3-piperidin-1-ylpropoxymethyl)-3-azabicyclo[3.1.0]hex-3-ylmethyl]-quinoxaline;
1-{4-[(1R,5S,6R)-6-(3-piperidin-1-ylpropoxymethyl)-3-azabicyclo[3.1.0]hex-3-ylmethyl]-phenyl}-imidazolidin-2-one;
(1R,5S,6R)-3-(4-benzyloxybenzyl)-6-(3-piperidin-1-ylpropoxymethyl)-3-azabicyclo[3.1.0]hexane;
(1R,5S,6R)-3-(4-pentyloxybenzyl)-6-(3-piperidin-1-ylpropoxymethyl)-3-azabicyclo[3.1.0]hexane;
(1R,5S,6R)-6-(3-piperidin-1-ylpropoxymethyl)-3-[4-(1H-tetrazol-5-yl)-benzyl]-3-azabicyclo[3.1.0]hexane;
3-(methyl-{4-[(1R,5S,6R)-6-(3-piperidin-1-ylpropoxymethyl)-3-azabicyclo[3.1.0]hex-3-ylmethyl]-phenyl}-amino)-propionitrile;
5-[(1R,5S,6R)-6-(3-piperidin-1-ylpropoxymethyl)-3-azabicyclo[3.1.0]hex-3-ylmethyl]-1H-indole;
(1R,5S,6R)-3-(4′-methoxybiphenyl-4-ylmethyl)-6-(3-piperidin-1-ylpropoxymethyl)-3-azabicyclo[3.1.0]hexane;
4-methyl-7-[(1R,5S,6R)-6-(3-piperidin-1-ylpropoxymethyl)-3-azabicyclo[3.1.0]hex-3-ylmethyl]-3,4-dihydro-2H-benzo[1,4]oxazine;
(1R,5S,6R)-3-[3-(cyclopent-3-enyloxy)-benzyl]-6-(3-piperidin-1-ylpropoxymethyl)-3-azabicyclo[3.1.0]hexane;
(1R,5S,6R)-6-(3-piperidin-1-ylpropoxymethyl)-3-[3-(1,1,2,2-tetrafluoroethoxy)-benzyl]-3-azabicyclo[3.1.0]hexane;
(1R,5S,6R)-3-(4-morpholin-4-ylbenzyl)-6-(3-piperidin-1-ylpropoxymethyl)-3-azabicyclo[3.1.0]hexane;
(1R,5S,6R)-3-[4-(cyclopent-3-enyloxy)-benzyl]-6-(3-piperidin-1-ylpropoxymethyl)-3-azabicyclo[3.1.0]hexane;
(1R,5S,6R)-6-(3-piperidin-1-ylpropoxymethyl)-3-(4-[1,2,4]triazol-1-yl benzyl)-3-azabicyclo[3.1.0]hexane;
2-{4-[(1R,5S,6R)-6-(3-piperidin-1-ylpropoxymethyl)-3-azabicyclo[3.1.0]hex-3-ylmethyl]-phenoxy}-acetamide;
(1R,5S,6R)-6-(3-piperidin-1-ylpropoxymethyl)-3-(4-pyrimidin-5-yl benzyl)-3-azabicyclo[3.1.0]hexane;
(1R,5S,6R)-3-(3-methoxybenzyl)-6-(3-piperidin-1-ylpropoxymethyl)-3-azabicyclo[3.1.0]hexane;
(1R,5S,6R)-6-(3-piperidin-1-ylpropoxymethyl)-3-(4-pyridin-3-ylbenzyl)-3-azabicyclo[3.1.0]hexane;
(1R,5S,6R)-3-(2,2-difluorobenzo[1,3]dioxol-5-ylmethyl)-6-[(3-piperidin-1-ylpropyl)oxymethyl]-3-azabicyclo[3.1.0]hexane;
(1R,5S,6R)-6-(3-piperidin-1-ylpropoxymethyl)-3-[4-(1,1,2,2-tetrafluoroethoxy)-benzyl]-3-azabicyclo[3.1.0]hexane;
(1R,5S,6R)-3-(4-isobutoxybenzyl)-6-(3-piperidin-1-ylpropoxymethyl)-3-azabicyclo[3.1.0]hexane;
(1R,5S,6R)-6-(3-piperidin-1-ylpropoxymethyl)-3-(4-pyrazol-1-ylbenzyl)-3-azabicyclo[3.1.0]hexane;
(1R,5S,6R)-3-(2-chlorobenzyl)-6-(3-piperidin-1-ylpropoxymethyl)-3-azabicyclo[3.1.0]hexane;
(1R,5S,6R)-3-(2,2-difluorobenzo[1,3]dioxol-4-ylmethyl)-6-[(3-piperidin-1-ylpropyl)oxymethyl]-3-azabicyclo[3.1.0]hexane;
(1R,5S,6R)-3-(2,3-dihydrobenzofuran-5-ylmethyl)-6-(3-piperidin-1-ylpropoxymethyl)-3-azabicyclo[3.1.0]hexane;
3-[(1S,5R,6R)-6-(3-piperidin-1-ylpropoxymethyl)-3-azabicyclo[3.1.0]hex-3-yl]-benzo[d]isoxazole;
3-phenethyl-6-(3-piperidin-1-ylpropoxymethyl)-3-azabicyclo[3.1.0]-hexane; and
3-(4-chlorobenzyl)-6-(3-piperidin-1-ylpropoxymethyl)-3-azabicyclo[3.1.0]hexane.

### DETAILED DESCRIPTION OF THE INVENTION

The compounds of formula I according to the invention may be prepared by the general procedure shown in Scheme 1-3.

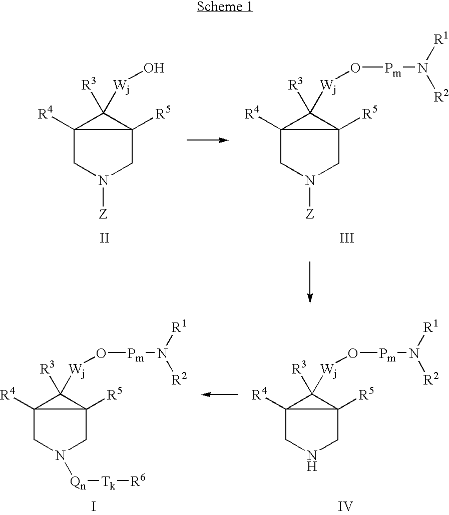

According to Scheme 1, an amine of the general formula II, in which the group Z is a protecting group, is reacted with an appropriately substituted compound of the general formula V:
L¹-Pₘ—NR¹R² (V)
wherein P, m, R¹ and R² are as defined above and L¹ is a leaving group selected from the list which includes (but is not limited to) Cl, Br, I, mesylate and tosylate to give an intermediate of the general formula III. The selection of a protecting group (Z) for this process will necessarily be influenced by the ease with which it can be removed in a subsequent step, but includes groups that have been effectively used to protect secondary amines, e.g., benzyl, tert-butoxycarbonyl (t-BOC), benzyloxycarbonyl (CBZ) and the like, as described by Theodora Greene and Peter Wuts in “Protecting Groups in Organic Synthesis”, 2^{nd} Ed., John-Wiley and Sons, Inc., NY, 1991, pp 309-385. The reaction is generally conducted under basic conditions to minimize the removal of the Z group, and may include the use of an organic base like pyridine, triethylamine (TEA) or trimethylamine (TMA) or an inorganic base like sodium or potassium bicarbonate or sodium or potassium carbonate in a reaction inert solvent such as THF, DMF, DMA or acetone. The reactions can be performed at temperatures in the range from about (−78)° C. to about the boiling point of the solvent selected for the reaction and at pressures from about one to about three atmospheres and are generally done under an inert atmosphere of nitrogen or argon gas at atmospheric pressure. The presence of a catalytic amount of potassium iodide (KI) may also increase the rate of the reaction, especially when L¹ is chlorine.

The protecting group Z of the intermediate of formula III can then be removed to produce the intermediate of general formula IV. A good resource for identifying the appropriate conditions to conduct this reaction is the Greene and Wuts reference. Included in this process are the uses of such reagents as dilute hydrochloric acid or methanesulfonic acid or sulfuric acid (in an organic solvent such as ethyl acetate, dioxane or THF), trifluoroacetic acid, trimethylsilyl iodide (in chloroform or acetonitrile) and others for the removal of the t-BOC group, and the use of catalytic hydrogenation, trimethylsilyl iodide in acetonitrile, boron tribromide in dichloromethane and other similar reagents for the removal of the CBZ protecting group.

The intermediate of formula IV can then be converted to a compound of general formula I using one of several different procedures, depending on the nature of the group Qₙ-Tₖ-R⁶. For example, reacting an aldehyde of the general formula VIII:
R⁶-Tₖ₋₁-CHO (VII)
with the intermediate of general formula IV can produce the product of formula I wherein n=0. This transformation is generally referred to as a reductive amination and can be performed under a variety of conditions known to one skilled in the art of chemistry. It may be completed in a single, concerted process (e.g., see A. F. Abdel-Magid, C. A. Maryanoff and K. G. Carson in Tetrahedron Letters, 1990, 39:5595-5598). In such conversions, the carbonyl compound of formula VII and the intermediate amine of formula IV are combined in a reaction inert solvent and treated with a reducing agent such as sodium cyanoborohydride (NaBH₃CN) or sodium triacetoxyborohydride (NaBH(OAc)₃). Suitable solvents include, among others, tetrahydrofuran (THF) and 1,2-dichloroethane (DCE) and the reaction may be conducted with or without the addition of an organic acid (e.g., acetic acid).

The conversion of compounds of formula IV into compounds of formula I can also be completed using two or more individual steps, e.g., involving the initial formation of an imine intermediate such as VIII, followed by reduction of the C═N double bond to generate the compounds of general formula I. In some instances, this intermediate can be isolated and purified.

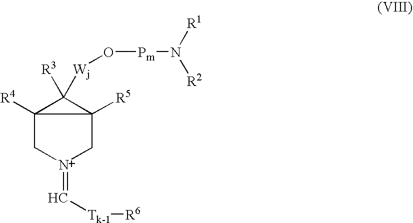

For example, the intermediate of formula IV and the appropriate aldehyde of formula VII can be combined in the presence of a dehydrating agent in a reaction inert solvent like benzene, toluene, methanol or ethanol and stirred for a prescribed amount of time until the reaction is judged to be complete (e.g., using techniques like thin layer chromatography (tic), mass spectrometry (MS) or nuclear magnetic resonance spectrometry (NMR) to monitor the progress of the reaction). Such dehydrating agents may include, for example, p-toluenesulfonic acid (i.e., PTSA), titanium(IV) chloride (i.e., TiCl₄), titanium(IV) isopropoxide (i.e., Ti(OiPr)₄) or molecular sieves. The reaction can be conducted within the range of about 0° C. to about the boiling point of the solvent employed and at pressures of about one to about three atmospheres. The intermediate imine VIII so obtained can then be reduced using one or more reducing agents under conditions familiar to one skilled in the art, e.g., hydrogen gas in the presence of a catalyst like palladium on carbon (Pd/C) or platinum on carbon (Pt/C), sodium borohydride (i.e., NaBH₄), sodium (triacetoxy)borohydride, sodium cyanoborohydride and the like. The use of hydrogen as the reducing agent is often conducted in a reaction inert solvent such as methanol, ethanol, THF, 1,4-dioxane or similar solvents at a pressure of about one atmosphere to a pressure of about five atmospheres of hydrogen and typically at a temperature from about room temperature to a temperature that is below or at the boiling point of the solvent employed. When using a hydride reagent, the choice of solvent can be made from, but not limited to, methanol, ethanol, isopropanol, 1,4-dioxane, THF and the like. The reaction can be carried out at atmospheric pressure and at temperatures ranging from about (40)° C. to about the boiling temperature of the solvent employed, typically at 0-40° C. and most preferably at room temperature.

Alternatively, compounds of the general formula I can be prepared from the intermediate of formula IV by an alkylation process, using a reagent of the general formula IX:
R⁶Tₖ-L³ (IX)
wherein R⁶, T and k are as previously defined and L³ is leaving group such as chlorine, bromine, iodine, mesylate, tosylate and the like. Conditions for these reactions are well known to those skilled in the art of organic chemistry and include combining the reactants of formulae IV and IX in a reaction inert solvent in the presence of an organic or inorganic base. Typical solvents for these reactions include those commonly used in organic synthesis, such as chloroform, dichloromethane, THF, dioxane, diethyl ether and the like, in the presence of a base such as sodium or potassium bicarbonate, sodium or potassium carbonate, sodium or potassium hydroxide, sodium hydride, trimethylamine (TMA) or triethylamine (TEA). Such reactions are typically performed at atmospheric pressure and at temperatures within the range of (80)° C. to about the boiling point of the solvent used.

The compounds of general formula I, wherein n is 1 and Q is a carbonyl (C═O) or sulfonyl (SO₂) can be prepared by reaction of the intermediate of general formula IV with a reagent such as X;
R⁶-Tₖ-Q-L⁴ (X)
wherein R⁶, T and k are as previously defined and L⁴ is leaving group, typically Cl or Br. As in the case for the alkylation of intermediate IV described above, these acylation and sulfonylation reactions can be conducted under similar conditions, i.e., combining IV and X in a reaction inert solvent in the presence of a base and stirring within the temperature range of (−80)° C. to about the boiling point of the solvent until the reaction is judged to be complete.

The preceding compounds of the general formula I, wherein n=1 and Q is carbonyl can be further converted to compounds wherein Q is CH₂ through the use of selective reducing agents. Such reducing agents may include lithium aluminum hydride (LAH) in diethyl ether or THF, or diborane in THF within the temperature range of (−80)° C. to about the boiling point of the solvent until the reaction is judged to be complete.

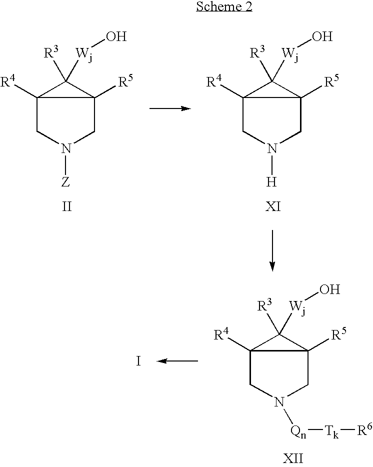

As shown in Scheme 2, the compounds of formula I may also be prepared using standard conditions for the formation of the ether bond as the final step in the sequence. Thus, a compound of the general formula II, wherein Z is a protecting group as previously defined, can be de-protected (i.e., the Z is removed) as previously described in the conversion of III to IV, to give the intermediate secondary amine of general formula XI. This intermediate can then be reacted with an appropriately substituted aldehyde of general formula VII, the appropriately substituted alkylating agent of general formula IX or the appropriately substituted carbonyl or sulfonyl derivative of general formula X: wherein Q, T, k, n, L³, L⁴ and R⁶ are as previously defined to produce an intermediate compound of general formula XII. This latter compound XII can then be converted, as described above for the conversion of III to IV, to give a compound of the general formula I.

In the examples that follow, the abbreviations used are intended to have the following, general meaning:
bm: broad multiplet (NMR)
bs: broad singlet (NMR)
dd: doublet of doublets (NMR)
d.e.: diatomaceous earth, filter agent
DMF: dimethylormamide
LRMS: low resolution mass spectrometry
calcd; calculated
d; doublet (NMR)
EtOAc: ethyl acetate
J: coupling constant (NMR)
LAH: lithium aluminum hydride
m: multiplet (in NMR)
min: minute(s)
m/z: mass to charge ratio (in mass spectrometry)
obsd: observed
Rf: retention factor (in chromatography)
Rt: retention time (in chromatography)
rt: room temperature
s: singlet (NMR), second(s)
t: triplet
THF: tetrahydrofuran
tlc: thin layer chromatography

Solvents were purchased and used without purification. Yields were calculated for material judged homogenous by thin layer chromatography and NMR. Thin layer chromatography was performed on Merck Kieselgel 60 F 254 plates eluting with the solvents indicated, visualized by a 254 nm UV lamp, and stained with either an aqueous KMnO₄ solution or an ethanolic solution of 12-molybdophosphoric acid. Flash column chromatography was performed with using either pre-packed Biotage® or ISCO® columns using the size indicated. Nuclear magnetic resonance (NMR) spectra were acquired on a Unity 400 or 500 at 400 MHz or 500 MHz for ¹H, respectively, and 100 MHz or 125 MHz for ¹³C NMR, respectively. Chemical shifts for proton ¹H NMR spectra are reported in parts per million relative to the singlet of CDCl₃ at 7.24 ppm. Chemical shifts for ¹³C NMR spectra are reported in parts per million downfield relative to the centerline of the triplet of CDCl₃ at 77.0 ppm. Mass spectra analyses were performed on a APCI Gilson 215, micromass ZMD (50% Acetonitrile/50% water) spectrometer.

Reactions under microwave conditions were done using 2-5 mL round bottom vials, fitted with septa. The vials containing the reactants were inserted into the reaction chamber of a EMRYS™ Creator microwave apparatus (maximum power of 300 W) from Personal Chemistry Inc., 25 Birch St., Bldg C, Suite 304, Milford, Mass. 01757 and heated to the appropriate temperature for a the prescribed period of time. HPLC was performed according to the following methods:

Method A: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 100% solvent A, 0% solvent B, flow 20; time 2.0, 100% solvent A, 0% solvent B, flow 20; time 12.0, 0% solvent A, 100% solvent B, flow 20; time 15.0, 0% solvent A, 100% solvent B, flow 20; time 15.1, 100% solvent A, 0% solvent B, flow 20; time 20.0, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splifter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method B: Preparative conditions (Waters 600 & Waters 2767 Sample Manager); Column: Waters Xterra PrepMS C₁₈ column, 5 μm, 30×150 mm steel column, part # 186001120, serial # T22881T 09; solvent A—0.1% Trifluoroacetic acid/water; solvent B—Acetonitrile; volume of injection: 1050 μL; time 0.0, 100% solvent A, 0% solvent B, flow 20; time 2.0, 100% solvent A, 0% solvent B, flow 20; time 12.0, 0% solvent A, 100% solvent B, flow 20; time 14.0, 0% solvent A, 100% solvent B, flow 20; time 14.1, 100% solvent A, 0% solvent B, flow 20; time 19.1, 100% solvent A, 0% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; Ion Energy: 0.2; Multiplier: 550.

Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

Method C: Preparative conditions (Waters 600 & Waters 2767 Sample Manager); Column: Waters Symmetry C₁₈, 5 μm, 30×150 mm steel column, part # WAT248000, serial # M12921A01; solvent A—0.1% Trifluoroacetic acid/water; solvent B—Acetonitrile; volume of injection: 850 μL; time 0.0, 90% solvent A, 10% solvent B, flow 20; time 10.0, 0% solvent A, 100% solvent B, flow 20; time 12.0, 0% solvent A, 100% solvent B, flow 20.

Mass spectral (micromassZO) conditions; Capillary(kV): 3.0; Cone (V): 20; Extractor (V): 3.0; RF Lens (V): 0.5; Source temp. (° C.): 120; Desolvation temp. (° C.): 360; Desolvation gas flow (L/hr): 450; Cone gas flow (L/hr): 150; LM Resolution: 15; HM Resolution: 15; [on Energy: 0.2; Multiplier: 550. Splitter; Acurate by LC Packings, 1/10,000; Upchurch needle valve setting: 14; Make up pump (Waters 515) Flow (ml/min.): 1. PDA (Waters 996) Settings; Start/End wavelength (nm): 200/600; Resolution: 1.2; Sample Rate: 1; Channels: TIC, 254 nm and 220 nm.

The following intermediates may be prepared by the procedures described above:

### Intermediate 1

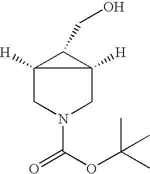

### (1S,5R,6R)-6-Hydroxymethyl-3-azabicyclo[3.1.0]hexane-3-carboxylic acid tert-butyl ester

(1S,5R,6R)-(3-Azabicyclo[3.1.0]hex-6-yl)-methanol (1.75 mg, 1.55 mmol) prepared according to the procedure of K. Brighty and M. Castaldi (Synlett, 1996, 11:1097-1099 was dissolved in 3 mL dichloromethane and treated with triethylamine (0.43 mL), resulting in a clear solution. This solution was then treated with di-tert-butyl dicarbonate (507 mg, 2.33 mmol) and stirred at rt for 72 hr. The reaction mixture was washed with saturated NaHCO₃ solution, saturated aqueous NaCl and dried with Na₂SO₄, then concentrated in vacuo to give a light brown syrup, 290 mg.

Mass spectrum (m/z) calcd for C₁₁H₁₉NO₃: 213.27; obsd. 214 (M+, 12%), 199 (100%).

### Intermediate 2

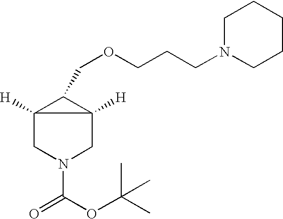

### (1S,5R,6R)-6-(3-Piperidin-1-ylpropoxymethyl)-3-azabicyclo[3.1.0]hexane-3-carboxylic acid tert-butyl ester

(1S,5R,6R)-6-Hydroxymethyl-3-azabicyclo[3.1.0]hexane-3-carboxylic acid tert-butyl ester (0.290 mg, 1.36 mmol) in 10 mL of THF was treated with 0.95 mL of 1M potassium tert-butoxide solution and warmed at 50° C. for 45 min. N-(3-chloropropyl)-piperidine (243 mg, 0.95 mmol of free base) was added and stirring was continued overnight at 80° C. After cooling to rt, the solvent was removed in vacuo, the residue diluted with EtOAc and water and the organic layer washed with additional water, saturated NaCl and dried with Na₂SO₄. Removal of the solvent in vacuo gave a tan syrup which was chromatographed using a gradient system of 5% CH₃OH:CH₂Cl₂ to 0.5% NH₄OH:5% CH₃OH:CH₂Cl₂ on a Biotage silica gel column to give a clear syrup, 400 mg.

Mass spectrum (m/z) calcd for C₁₉H₃₄N₂O₃: 338.42; obsd. 339 (M+1, 100%).

### Intermediate 3

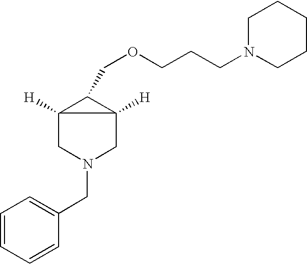

### (1S,5R,6R)-3-Benzyl-6-(3-piperidin-1-ylpropoxymethyl)-3-azabicyclo[3.1.0]hexane

In the same manner as described in Intermediate 2, (1S,5R,6R)-(3-benzyl-3-azabicyclo[3.1.0]hex-6-yl)-methanol (1.91 g, 6.3 mmol, prepared according to the method of Brighty) in 25 mL of THF was treated with 6.93 mL of 1M potassium tert-butoxide, followed by N-(3-chloropropyl)-piperidine (1.12 g, 6.93 mmol) to give 723 mg of the desired product as a golden syrup after chromatographic purification.

Mass spectrum (m/z) calcd for C₂₁H₃₂N₂O: 328.41; obsd. 329 (M+1, 100%).

¹H-nmr (CDCl₃, 400 MHz) δ 1.26 (m, 2H), 1.42 (m, 2H), 1.52-1.60 (m, 5H), 1.69-1.80 (m, 2H), 2.32-2.37 (m, 9H), 2.98 (d, 2H), 3.23 (d, 2H), 3.44 (t, 2H), 3.57 (s, 2H), 7.21-7.29 (m, 5H).

### Intermediate 4

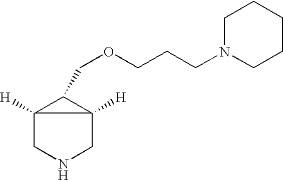

### (1S,5R,6R)-6-(3-Piperidin-1-ylpropoxymethyl)-3-azabicyclo[3.1.0]hexane

(1S,5R,6R)-6-(3-Piperidin-1-ylpropoxymethyl)-3-azabicyclo[3.1.0]hexane-3-carboxylic acid tert-butyl ester (400 mg, Intermediate 2) in 15 mL CH₃OH was treated with 1 mL of 4N HCl in dioxane and stirred for 18 hr at 50° C. The clear solution was then concentrated in vacuo, dissolved in water and washed with CH₂Cl₂. The aqueous layer was then adjusted to pH 14 with 2N NaOH and extracted with CH₂Cl₂. This organic layer was then washed with saturated NaCl and dried with Na₂SO₄, then concentrated in vacuo to give 220 mg of a clear oil.

Mass spectrum (m/z) calcd for C₁₄H₂₆N₂O: 238.37; obsd. 239 (M+1, 100%).

¹H-nmr (CDCl₃, 400 MHz) δ 0.82 (m, 1H), 1.30 (s, 2H), 1.41 (bs, 2H), 1.57 (m, 5H), 1.76 (m, 2H), 2.34-2.42 (m, 6H), 2.82 (d, 2H), 2.97 (d, 2H), 3.28 (d, 2H), 3.43 (d, 2H).

Alternatively, this intermediate was prepared by hydrogenating (1S,5R,6R)-3-benzyl-6-(3-piperidin-1-yl-propoxymethyl)-3-aza-bicyclo[3.1.0]hexane (400 mg, intermediate 3) with 75 mg of palladium hydroxide in 20 mL CH₃OH in a Parr shaker apparatus at 45 psi for 3 hr at rt.

The following compounds may be prepared by the procedures below:

### EXAMPLE 1

### General Procedure A

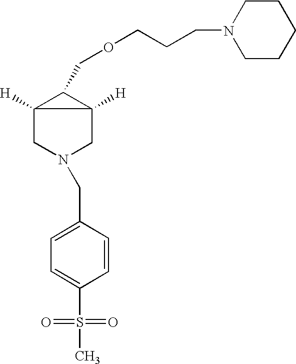

### (1S,5R,6R)-3-(4-Methanesulfonylbenzyl)-6-(3-piperidin-1-ylpropoxymethyl)-3-azabicyclo[3.1.0]-hexane

(1S,5R,6R)-6-(3-Piperidin-1-ylpropoxymethyl)-3-azabicyclo[3.1.0]hexane (110 mg, 0.46 mmol, intermediate 4) in 3 mL acetic acid was treated with 4-(methanesulfonyl)-benzaldehyde (255 mg, 1.38 mmol) and stirred at rt for 1 hr. Sodium triacetoxyborohydride (390 mg, 1.84 mmol) was added portionwise over 15 min and the reaction stirred at rt for 48 hr. The mixture was diluted with dilute aqueous NaHCO₃ and extracted with EtOAc, the organic layer was washed with water, saturated NaCl and dried with Na₂CO₃. Removal of the solvent in vacuo gave a crude oil which after silica gel chromatography produced 33 mg of pale yellow oil. This was converted to the dihydrochloride salt by dissolving it in the minimal volume of EtOAc and treating with 1M HCl in diethyl ether (Aldrich Chemical Co.).

Mass spectrum (m/z) calcd for C₂₂H₃₄N₃O₃S: 406.59; obsd. 407.2 (M+1, 100%), 408.3 (35%).

¹H-nmr (CDCl₃, 400 MHz) δ 1.27 (m, 2H), 1.44 (bs, 2H), 1.49 (m, 1H), 1.64 (bs, 4H), 1.82 (bs, 2H), 2.34 (d, 2H), 2.46 (bs, 6H), 2.96 (d, 2H), 3.02 (s, 3H), 3.22 (d, 2H), 3.44 (t, 2H), 3.63 (s, 2H), 7.45 (m, 2H), 7.82 (m, 2H).

The following compounds were also prepared using the general procedure A, as described for Example 1:

### EXAMPLE 2

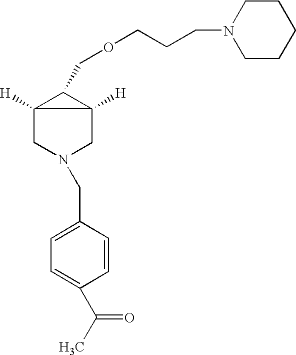

### (1S,5R,6R)-1-{4-[6-(3-Piperidin-1-ylpropoxymethyl)-3-azabicyclo[3.1.0]hex-3-ylmethyl]-phenyl}-ethanone

Mass spectrum (m/z) calcd for C₂₃H₃₄N₂O₂: 370.53; obsd. 371.2 (M+1,100%), 372 (45%), 373 (27%).

¹H-nmr (CDCl₃, 400 MHz) 61.25 (m, 2H), 1.49 (bs, 2H), 1.53 (m, 1H), 1.63 (bs, 4H), 1.82 (bs, 2H), 2.32 (dd, 2H), 2.42 (bs, 6H), 2.56 (s, 3H), 2.96 (d, 2H), 3.21 (d, 2H), 3.43 (t, 2H), 3.60 (s, 2H), 7.33 (d, 2H), 7.85 (dd, 2H).

### EXAMPLE 3

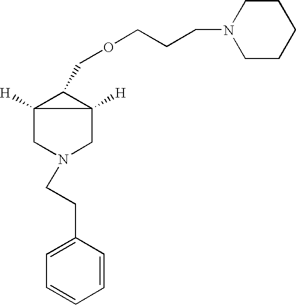

### (1S,5R,6R)-3-Phenethyl-6-(3-piperidin-1-ylpropoxymethyl)-3-azabicyclo[3.1.0]-hexane

Mass spectrum (m/z) calcd for C₂₂H₃₄N₂O: 342.53; obsd. 343.2 (M+1, 100%), 344 (22%).

¹H-nmr (CDCl₃, 400 MHz) δ 1.24 (m, 2H), 1.41 (m, 2H), 1.55 (bs, 5H), 1.63 (bs, 2H), 1.73 (m, 2H), 2.33 (m, 8H), 2.63 (m, 2H), 3.08 (m, 2H), 3.21 (bs, 2H), 3.43 (bs, 2H), 7.15-7.26 (bm, 5H).

### EXAMPLE 4

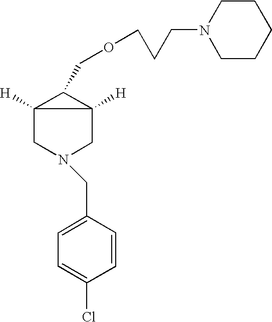

### (1S,5R,6R)-3-(4-Chlorobenzyl)-6-(3-Piperidin-1-ylpropoxymethyl)-3-azabicyclo[3.1.0]hexane

Mass spectrum (m/z) calcd for C₂₁H₃₁ClN₂O: 362.94; obsd. 363.2 (M+1, 100%).

¹H-nmr (CDCl₃, 400 MHz) δ 1.23 (m, 2H), 1.47 (m, 1H), 1.57 (bs, 1H), 1.92 (bm, 5H), 2.06 (bm, 2H), 2.30 (d, 2H), 2.87 (bm, 6H), 2.93 (d, 2H), 3.21 (d, 2H), 3.47 (t, 2H), 3.51 (s, 2H), 7.16-7.23 (m, 4H).

### EXAMPLE 5

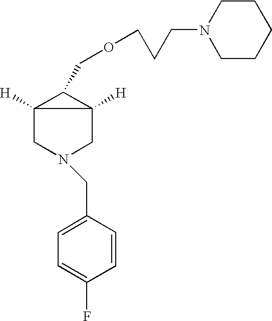

### (1S,5R,6R)-3-(4-Fluorobenzyl)-6-(3-piperidin-1-ylpropoxymethyl)-3-azabicyclo[3.1.0]hexane

Mass spectrum (m/z) calcd for C₂₁H₃₁FN₂O: 346.49; obsd. 347.2 (M+1, 100%).

¹H-nmr (CDCl₃, 400 MHz) δ 1.23 (m, 2H), 1.45-1.50 (m, 2H), 1.74 (bm, 5H), 1.91 (bm, 2H), 2.30 (d, 2H), 2.61 (bm, 6H), 2.92 (d, 2H), 3.21 (d, 2H), 3.45 (t, 2H), 3.51 (s, 2H), 6.91-6.96 (m, 2H), 7.17-7.21 (m, 2H).

### EXAMPLE 6

### (1S,5R,6R)-3-naphthalen-2-ylmethyl-6-(3-piperidin-1-ylpropoxymethyl)-3-azabicyclo[3.1.0]hexane

Mass spectrum (m/z) calcd for C₂₅H₃₄N₂O: 378.56; obsd. 379.2 (M+1, 100%).

### EXAMPLE 7

### (1S,5R,6R)-6-(3-piperidin-1-ylpropoxymethyl)-3-pyridin-3-ylmethyl-3-azabicyclo[3.1.0]hexane

Mass spectrum (m/z) calcd for C₂₀H₃₁N₃O: 329.48; obsd. 330.2 (M+1, 100%).

### EXAMPLE 8

### (1S,5R,6R)-3-(4-methoxybenzyl)-6-(3-piperidin-1-ylpropoxymethyl)-3-azabicyclo[3.1.0]hexane

Mass spectrum (m/z) calcd for C₂₂H₃₄N₂O₂: 358.52; obsd. 359.2 (M+1, 100%).

### EXAMPLE 9

### (1S,5R,6R)-3-(4-methylbenzyl)-6-(3-piperidin-1-ylpropoxymethyl)-3-azabicyclo[3.1.0]hexane

Mass spectrum (m/z) calcd for C₂₂H₃₄N₂O: 342.52; obsd. 343.2 (M+1, 100%).

### EXAMPLE 10

### (1S,5R,6R)-3-(3-fluorobenzyl)-6-(3-piperidin-1-ylpropoxymethyl)-3-azabicyclo[3.1.0]hexane

Mass spectrum (m/z) calcd for C₂₁H₃₁FN₂O: 346.49; obsd. 347.2 (M+1, 100%).

### EXAMPLE 11

### 3-[(1S,5R,6R)-6-(3-piperidin-1-ylpropoxymethyl)-3-azabicyclo[3.1.0]hex-3-ylmethyl]-phenol

Mass spectrum (m/z) calcd for C₂₁H₃₂N₂O₂: 344.50; obsd. 345.2 (M+1, 100%).

### EXAMPLE 12

### 4-[(1S,5R,6R)-6-(3-piperidin-1-ylpropoxymethyl)-3-azabicyclo[3.1.0]hex-3-ylmethyl]-phenol

Mass spectrum (m/z) calcd for C₂₁H₃₂N₂O₂: 344.50; obsd. 345.2 (M+1, 100%).

### EXAMPLE 13

### 4-[(1S,5R,6R)-6-(3-piperidin-1-ylpropoxymethyl)-3-azabicyclo[3.1.0]hex-3-ylmethyl]-benzonitrile

Mass spectrum (m/z) calcd for C₂₂H₃₁N₃O: 353.51; obsd. 354.2 (M+1, 100%).

### EXAMPLE 14

### (1S,5R,6R)-3-(3-phenoxybenzyl)-6-(3-piperidin-1-ylpropoxymethyl)-3-azabicyclo[3.1.0]hexane

Mass spectrum (m/z) calcd for C₂₇H₃₆N₂O₂: 420.59; obsd. 421.2 (M+1, 100%).

### EXAMPLE 15

### (1S,5R,6R)-3-(3-benzyloxybenzyl)-6-(3-piperidin-1-ylpropoxymethyl)-3-azabicyclo[3.1.0]hexane

Mass spectrum (m/z) calcd for C₂₈H₃₈N₂O₂: 434.62; obsd. 435.2 (M+1, 100%).

### EXAMPLE 16

### (1S,5R,6R)-3-(4-butoxybenzyl)-6-(3-piperidin-1-ylpropoxymethyl)-3-azabicyclo[3.1.0]hexane

Mass spectrum (m/z) calcd for C₂₅H₄₀N₂O₂: 400.60; obsd. 401.2 (M+1, 100%).

### EXAMPLE 17

### (1S,5R,6R)-3-biphenyl-4-ylmethyl-6-(3-piperidin-1-ylpropoxymethyl)-3-azabicyclo[3.1.0]hexane

Mass spectrum (m/z) calcd for C₂₇H₃₆N₂O: 404.59; obsd. 405.2 (M+1, 100%).

### EXAMPLE 18

### (1S,5R,6R)-3-benzo[1,3]dioxol-5-ylmethyl-6-(3-piperidin-1-ylpropoxymethyl)-3-azabicyclo[3.1.0]hexane

Mass spectrum (m/z) calcd for C₂₂H₃₂N₂O₃: 372.51; obsd. 373.2 (M+1, 100%).

### EXAMPLE 19

### (1S,5R,6R)-6-(3-piperidin-1-ylpropoxymethyl)-3-(3-trifluoromethylbenzyl)-3-azabicyclo[3.1.0]hexane

Mass spectrum (m/z) calcd for C₂₂H₃₁F₃N₂O: 396.49; obsd. 397.2 (M+1, 100%).

### EXAMPLE 20

### (1S,5R,6R)-3-(4-bromobenzyl)-6-(3-piperidin-1-ylpropoxymethyl)-3-azabicyclo[3.1.0]hexane

Mass spectrum (m/z) calcd for C₂₁H₃₁BrN₂O: 407.39; obsd. 408.2 (M+1, 100%).

### EXAMPLE 21

### (1S,5R,6R)-3-(4-isopropylbenzyl)-6-(3-piperidin-1-ylpropoxymethyl)-3-azabicyclo[3.1.0]hexane

Mass spectrum (m/z) calcd for C₂₄H₃₈N₂O: 370.58; obsd. 371.2 (M+1, 100%).

### EXAMPLE 22

### (1S,5R,6R)-3-(3-chlorobenzyl)-6-(3-piperidin-1-ylpropoxymethyl)-3-azabicyclo[3.1.0]hexane

Mass spectrum (m/z) calcd for C₂₁H₃₁ClN₂O: 362.94; obsd. 363.2 (M+1, 100%).

### EXAMPLE 23

### (1S,5R,6R)-3-(2,3-dihydro-benzo[1,4]dioxin-6-ylmethyl)-6-(3-piperidin-1-ylpropoxymethyl)-3-azabicyclo[3.1.0]hexane

Mass spectrum (m/z) calcd for C₂₃H₃₄N₂O₃: 386.53; obsd. 387.2 (M+1, 100%).

### EXAMPLE 24

### (1S,5R,6R)-3-(4-ethoxybenzyl)-6-(3-piperidin-1-ylpropoxymethyl)-3-azabicyclo[3.1.0]hexane

Mass spectrum (m/z) calcd for C₂₃H₃₁N₂O₂: 372.55; obsd. 373.2 (M+1, 100%).

### EXAMPLE 25

### (1S,5R,6R)-3-(4-tert-butylbenzyl)-6-(3-piperidin-1-ylpropoxymethyl)-3-azabicyclo[3.1.0]hexane

Mass spectrum (m/z) calcd for C₂₅H₄₀N₂O: 384.60; obsd. 385.2 (M+1, 100%).

### EXAMPLE 26

### 3-[(1S,5R,6R)-6-(3-piperidin-1-ylpropoxymethyl)-3-azabicyclo[3.1.0]hex-3-ylmethyl]-benzonitrile

Mass spectrum (m/z) calcd for C₂₂H₃₁N₃O: 353.51; obsd. 354.2 (M+1,100%).

### EXAMPLE 27

### (1S,5R,6R)-3-(3,5-dichlorobenzyl)-6-(3-piperidin-1-ylpropoxymethyl)-3-azabicyclo[3.1.0]hexane

Mass spectrum (m/z) calcd for C₂₁H₃₀Cl₂N₂O: 397.39; obsd. 398.2 (M+1, 100%).

### EXAMPLE 28

### (1S,5R,6R)-3-benzo[1,3]dioxol-4-ylmethyl-6-(3-piperidin-1-ylpropoxymethyl)-3-azabicyclo[3.1.0]hexane

Mass spectrum (m/z) calcd for C₂₂H₃₂N₂O₃: 372.51; obsd. 373.2 (M+1, 100%).

### EXAMPLE 29

### (1S,5R,6R)-6-(3-piperidin-1-ylpropoxymethyl)-3-(4-trifluoromethylbenzyl)-3-azabicyclo[3.1.0]hexane

Mass spectrum (m/z) calcd for C₂₂H₃₁F₃N₂O: 396.49; obsd. 397.2 (M+1, 100%).

### EXAMPLE 30

### (1S,5R,6R)-3-(4-phenoxybenzyl)-6-(3-piperidin-1-ylpropoxymethyl)-3-azabicyclo[3.1.0]hexane

Mass spectrum (m/z) calcd for C₂₇H₃₆N₂O₂: 420.59; obsd. 421.2 (M+1, 100%).

### EXAMPLE 31

### (1S,5R,6R)-3-(2,6-difluorobenzyl)-6-(3-piperidin-1-ylpropoxymethyl)-3-azabicyclo[3.1.0]hexane

Mass spectrum (m/z) calcd for C₂₁H₃₀F₂N₂O: 364.48; obsd. 365.2 (M+1, 100%).

### EXAMPLE 32

### (1S,5R,6R)-3-(4-methylsulfanylbenzyl)-6-(3-piperidin-1-ylpropoxymethyl)-3-azabicyclo[3.1.0]hexane

Mass spectrum (m/z) calcd for C₂₂H₃₄N₂OS: 374.59; obsd. 375.2 (M+1, 100%).

### EXAMPLE 33

### (1R,5S,6R)-3-(3,5-difluorobenzyl)-6-[(3-piperidin-1-ylpropyl)oxymethyl]-3-azabicyclo[3.1.0]hexane

Mass spectrum (m/z) calcd for C₂₁H₃₀F₂N₂O: 364.48; obsd. 365.2 (M+1, 100%).

### EXAMPLE 34

### (1R,5S,6R)-6-(3-piperidin-1-ylpropoxymethyl)-3-(4-propoxybenzyl)-3-azabicyclo[3.1.0]hexane

Mass spectrum (m/z) calcd for C₂₄H₃₈N₂O₂: 386.58; obsd. 387.2 (M+1, 100%).

### EXAMPLE 35

### (1R,5S,6R)-3-(4-fluoro-3-trifluoromethylbenzyl)-6-[(3-piperidin-1-ylpropyl)oxymethyl]-3-azabicyclo[3.1.0]hexane

Mass spectrum (m/z) calcd for C₂₂H₃₀F₄N₂O: 414.48; obsd. 415.2 (M+1, 100%).

### EXAMPLE 36

### (1R,5S,6R)-3-(4-tert-butoxybenzyl)-6-(3-piperidin-1-ylpropoxymethyl)-3-azabicyclo[3.1.0]hexane

Mass spectrum (m/z) calcd for C₂₅H₄₀N₂O₂: 400.60; obsd. 401.2 (M+1, 100%).

### EXAMPLE 37

### 5-[(1R,5S,6R)-6-(3-piperidin-1-ylpropoxymethyl)-3-azabicyclo[3.1.0]hex-3-ylmethyl]-benzene-1,3-diol

Mass spectrum (m/z) calcd for C₂₁H₃₂N₂O₃: 360.49; obsd. 361.2 (M+1, 100%).

### EXAMPLE 38

### (1R,5S,6R)-6-(3-piperidin-1-ylpropoxymethyl)-3-(4-trifluoromethoxybenzyl)-3-azabicyclo[3.1.0]hexane

Mass spectrum (m/z) calcd for C₂₂H₃₁F₃N₂O₂: 412.49; obsd. 413.2 (M+1, 100%).

### EXAMPLE 39

### (1R,5S,6R)-3-(3-ethoxy-4-methoxybenzyl)-6-[(3-piperidin-1-ylpropyl)oxymethyl]-3-azabicyclo[3.1.0]hexane

Mass spectrum (m/z) calcd for C₂₄H₃₈N₂O₃: 402.58; obsd. 403.2 (M+1, 100%).

### EXAMPLE 40

### (1R,5S,6R)-6-(3-piperidin-1-ylpropoxymethyl)-3-(4-trifluoromethylsulfanylbenzyl)-3-azabicyclo[3.1.0]hexane

Mass spectrum (m/z) calcd for C₂₂H₃₁F₃N₂OS: 428.56; obsd. 429.2 (M+1, 100%).

### EXAMPLE 41

### (1R,5S,6R)-3-(4-ethylbenzyl)-6-(3-piperidin-1-ylpropoxymethyl)-3-azabicyclo[3.1.0]hexane

Mass spectrum (m/z) calcd for C₂₃H₃₆N₂O: 356.55; obsd. 357.2 (M+1, 100%).

### EXAMPLE 42

### (1R,5S,6R)-3-(4-isopropoxybenzyl)-6-(3-piperidin-1-ylpropoxymethyl)-3-azabicyclo[3.1.0]hexane

Mass spectrum (m/z) calcd for C₂₄H₃₈N₂O₂: 386.58; obsd. 387.2 (M+1, 100%).

### EXAMPLE 43

### (1R,5S,6R)-3-(3,5-dimethylbenzyl)-6-[(3-piperidin-1-ylpropyl)oxymethyl]-3-azabicyclo[3.1.0]hexane

Mass spectrum (m/z) calcd for C₂₃H₃₆N₂O: 356.55; obsd. 357.2 (M+1, 100%).

### EXAMPLE 44

### (1R,5S,6R)-3-(2′-methylbiphenyl-4-ylmethyl)-6-(3-piperidin-1-ylpropoxymethyl)-3-azabicyclo[3.1.0]hexane

Mass spectrum (m/z) calcd for C₂₈H₃₈N₂O: 418.62; obsd. 419.2 (M+1, 100%).

### EXAMPLE 45

### 2-{4-[(1R,5S,6R)-6-(3-piperidin-1-ylpropoxymethyl)-3-azabicyclo[3.1.0]hex-3-ylmethyl]-phenoxy}-ethanol

Mass spectrum (m/z) calcd for C₂₃H₃₆N₂O₃: 388.55; obsd. 389.2 (M+1, 100%).

### EXAMPLE 46

### (1R,5S,6R)-3-(4-isobutylbenzyl)-6-(3-piperidin-1-ylpropoxymethyl)-3-azabicyclo[3.1.0]hexane

Mass spectrum (m/z) calcd for C₂₅H₄₀N₂O: 384.60; obsd. 385.2 (M+1, 100%).

### EXAMPLE 47

### (1R,5S,6R)-3-[4-(4-fluorophenoxy)-benzyl]-6-(3-piperidin-1-ylpropoxymethyl)-3-azabicyclo[3.1.0]hexane

Mass spectrum (m/z) calcd for C₂₇H₃₅FN₂O₂: 438.58; obsd. 439.2 (M+1, 100%).

### EXAMPLE 48

### (1R,5S,6R)-3-(2,2-dimethylchroman-6-ylmethyl)-6-[(3-piperidin-1-ylpropyl)oxymethyl]-3-azabicyclo[3.1.0]hexane

Mass spectrum (m/z) calcd for C₂₆H₄₀N₂O₂: 412.61; obsd. 413.2 (M+1, 100%).

### EXAMPLE 49

### N-{4-[(1R,5S,6R)-6-(3-piperidin-1-ylpropoxymethyl)-3-azabicyclo[3.1.0]hex-3-ylmethyl]-phenyl}-acetamide

Mass spectrum (m/z) calcd for C₂₃H₃₅N₃O₂: 385.55; obsd. 386.2 (M+1, 100%).

### EXAMPLE 50

### 6-[(1R,5S,6R)-6-(3-piperidin-1-ylpropoxymethyl)-3-azabicyclo[3.1.0]hex-3-ylmethyl]-quinoxaline

Mass spectrum (m/z) calcd for C₂₃H₃₂N₄O: 380.53; obsd. 381.2 (M+1, 100%).

### EXAMPLE 51

### 1-{4-[(1R,5S,6R)-6-(3-piperidin-1-ylpropoxymethyl)-3-azabicyclo[3.1.0]hex-3-ylmethyl]-phenyl}-imidazolidin-2-one

Mass spectrum (m/z) calcd for C₂₄H₃₆N₄O₂: 412.57; obsd. 413.2 (M+1, 100%).

### EXAMPLE 52

### (1R,5S,6R)-3-(4-benzyloxybenzyl)-6-(3-piperidin-1-ylpropoxymethyl)-3-azabicyclo[3.1.0]hexane

Mass spectrum (m/z) calcd for C₂₈H₃₈N₂O₂: 434.62; obsd. 435.2 (M+1, 100%).

### EXAMPLE 53

### (1R,5S,6R)-3-(4-pentyloxybenzyl)-6-(3-piperidin-1-ylpropoxymethyl)-3-azabicyclo[3.1.0]hexane

Mass spectrum (m/z) calcd for C₂₆H₄₂N₂O₂: 414.63; obsd. 415.2 (M+1, 100%).

### EXAMPLE 54

### (1R,5S,6R)-6-(3-piperidin-1-ylpropoxymethyl)-3-[4-(1H-tetrazol-5-yl)-benzyl]-3-azabicyclo[3.1.0]hexane

Mass spectrum (m/z) calcd for C₂₂H₃₂N₆O: 396.54; obsd. 397.2 (M+1, 100%).

### EXAMPLE 55

### 3-(methyl-{4-[(1R,5S,6R)-6-(3-piperidin-1-ylpropoxymethyl)-3-azabicyclo[3.1.0]hex-3-vimethyl]-phenyl}-amino)-propionitrile

Mass spectrum (m/z) calcd for C₂₅H₃₈N₄O: 410.60; obsd. 411.2 (M+1, 100%).

### EXAMPLE 56

### 5-[(1R,5S,6R)-6-(3-piperidin-1-ylpropoxymethyl)-3-azabicyclo[3.1.0]hex-3-ylmethyl]-1H-indole

Mass spectrum (m/z) calcd for C₂₃H₃₃N₃O: 367.53; obsd. 368.2 (M+1, 100%).

### EXAMPLE 57

### (1R,5S,6R)-3-(4′-methoxybiphenyl-4-ylmethyl)-6-(3-piperidin-1-ylpropoxymethyl)-3-azabicyclo[3.1.0]hexane

Mass spectrum (m/z) calcd for C₂₈H₃₈N₂O₂: 434.62; obsd. 435.2 (M+1, 100%).

### EXAMPLE 58

### 4-methyl-7-[(1R,5S,6R)-6-(3-piperidin-1-ylpropoxymethyl)-3-azabicyclo[3.1.0]hex-3-ylmethyl]-3,4-dihydro-2H-benzo[1,4]oxazine

Mass spectrum (m/z) calcd for C₂₄H₃₇N₃O₂: 399.58; obsd. 400.2 (M+1, 100%).

### EXAMPLE 59

### (1R,5S,6R)-3-[3-(cyclopent-3-enyloxy)-benzyl]-6-(3-piperidin-1-ylpropoxymethyl)-3-azabicyclo[3.1.0]hexane

Mass spectrum (m/z) calcd for C₂₆H₃₈N₂O₂: 410.60; obsd. 411.2 (M+1, 100%).

### EXAMPLE 60

### (1R,5S,6R)-6-(3-piperidin-1-ylpropoxymethyl)-3-[3-(1,1,2,2-tetrafluoroethoxy)-benzyl]-3-azabicyclo[3.1.0]hexane

Mass spectrum (m/z) calcd for C₂₃H₃₂F₄N₂O₂: 444.51; obsd. 445.2 (M+1, 100%).

### EXAMPLE 61

### (1R,5S,6R)-3-(4-morpholin-4-ylbenzyl)-6-(3-piperidin-1-ylpropoxymethyl)-3-azabicyclo[3.1.0]hexane

Mass spectrum (m/z) calcd for C₂₅H₃₉N₃O₂: 413.60; obsd. 414.2 (M+1, 100%).

### EXAMPLE 62

### (1R,5S,6R)-3-[4-(cyclopent-3-enyloxy)-benzyl]-6-(3-piperidin-1-ylpropoxymethyl)-3-azabicyclo[3.1.0]hexane

Mass spectrum (m/z) calcd for C₂₆H₃₈N₂O₂: 410.60; obsd. 411.2 (M+1, 100%).

### EXAMPLE 63

### (1R,5S,6R)-6-(3-piperidin-1-ylpropoxymethyl)-3-(4-[1,2,4]triazol-1-ylbenzyl)-3-azabicyclo[3.1.0]hexane

Mass spectrum (m/z) calcd for C₂₃H₃₃N₅O: 395.55; obsd. 396.2 (M+1, 100%).

### EXAMPLE 64

### 2-{4-[(1R,5S,6R)-6-(3-piperidin-1-ylpropoxymethyl)-3-azabicyclo[3.1.0]hex-3-ylmethyl]-phenoxy}-acetamide

Mass spectrum (m/z) calcd for C₂₃H₃₅N₃O₃: 401.55; obsd. 402.2 (M+1, 100%).

### EXAMPLE 65

### (1R,5S,6R)-6-(3-piperidin-1-ylpropoxymethyl)-3-(4-pyrimidin-5-ylbenzyl)-3-azabicyclo[3.1.0]hexane

Mass spectrum (m/z) calcd for C₂₅H₃₄N₄O: 406.57; obsd. 407.2 (M+1, 100%).

### EXAMPLE 66

### (1R,5S,6R)-3-(3-methoxybenzyl)-6-(3-piperidin-1-ylpropoxymethyl)-3-azabicyclo[3.1.0]hexane

Mass spectrum (m/z) calcd for C₂₂H₃₄N₂O₂: 358.52; obsd. 359.2 (M+1, 100%).

### EXAMPLE 67

### (1R,5S,6R)-6-(3-piperidin-1-ylpropoxymethyl)-3-(4-pyridin-3-ylbenzyl)-3-azabicyclo[3.1.0]hexane

Mass spectrum (m/z) calcd for C₂₆H₃₅N₃O: 405.58; obsd. 406.2 (M+1, 100%).

### EXAMPLE 68

### (1R,5S,6R)-3-(2,2-difluorobenzo[1,3]dioxol-5-ylmethyl)-6-[(3-piperidin-1-ylpropyl)oxymethyl]-3-azabicyclo[3.1.0]hexane

Mass spectrum (m/z) calcd for C₂₂H₃₀F₂N₂O₃: 408.49; obsd. 409.2 (M+1, 100%).

### EXAMPLE 69

### (1R,5S,6R)-6-(3-piperidin-1-ylpropoxymethyl)-3-[4-(1,1,2,2-tetrafluoroethoxy)-benzyl]-3-azabicyclo[3.1.0]hexane

Mass spectrum (m/z) calcd for C₂₃H₃₂F₄N₂O₂: 444.51; obsd. 445.2 (M+1, 100%).

### EXAMPLE 70

### (1R,5S,6R)-3-(4-isobutoxybenzyl)-6-(3-piperidin-1-ylpropoxymethyl)-3-azabicyclo[3.1.0]hexane

Mass spectrum (m/z) calcd for C₂₅H₄₀N₂O₂: 400.60; obsd. 401.2 (M+1, 100%).

### EXAMPLE 71

### (1R,5S,6R)-6-(3-piperidin-1-ylpropoxymethyl)-3-(4-pyrazol-1-ylbenzyl)-3-azabicyclo[3.1.0]hexane

Mass spectrum (m/z) calcd for C₂₄H₃₄N₄O: 394.56; obsd. 395.2 (M+1, 100%).

### EXAMPLE 72

### (1R,5S,6R)-3-(2-chlorobenzyl)-6-(3-piperidin-1-ylpropoxymethyl)-3-azabicyclo[3.1.0]hexane

Mass spectrum (m/z) calcd for C₂₁H₃₁ClN₂O: 362.94; obsd. 363.2 (M+1, 100%).

### EXAMPLE 73

### (1R,5S,6R)-3-(2,2-difluorobenzo[1,3]dioxol-4-ylmethyl)-6-[(3-piperidin-1-ylpropyl)oxymethyl]-3-azabicyclo[3.1.0]hexane

Mass spectrum (m/z) calcd for C₂₂H₃₀F₂N₂O₃: 408.49; obsd. 409.2 (M+1, 100%).

### EXAMPLE 74

### (1R,5S,6R)-3-(2,3-dihydrobenzofuran-5-ylmethyl)-6-(3-piperidin-1-ylpropoxymethyl)-3-azabicyclo[3.1.0]hexane

Mass spectrum (m/z) calcd for C₂₃H₃₄N₂O₂: 370.53; obsd. 371.2 (M+1, 100%).

### EXAMPLE 75

### 3-[(1S,5R,6R)-6-(3-piperidin-1-ylpropoxymethyl)-3-azabicyclo[3.1.0]hex-3-yl]-benzo[d]isoxazole

Mass spectrum (m/z) calcd for C₂₁H₂₉N₃O₂: 355.48; obsd. 356.2 (M+1, 100%).

### Determination of Biological Activity

The in vitro affinity of the compounds in the present invention at the rat or human histamine H3 receptors can be determined according to the following procedure. Frozen rat frontal brain or frozen human post-mortem frontal brain is homogenized in 20 volumes of cold 50 mM Tris HCl containing 2 mM MgCl₂ (pH to 7.4 at 4° C.). The homogenate is then centrifuged at 45,000 G for 10 minutes. The supernatant is decanted and the membrane pellet re-suspended by Polytron in cold 50 mM Tris HCl containing 2 mM MgCl₂ (pH to 7.4 at 4 degrees C.) and centrifuged again. The final pellet is re-suspended in 50 mM Tris HCl containing 2 mM MgCl₂ (pH to 7.4 at 25 degrees C.) at a concentration of 12 mg/mL. Dilutions of compounds are made in 10% DMSO/50 mM Tris buffer (pH 7.4) (at 10× final concentration, so that the final DMSO concentration is 1%). Incubations are initiated by the addition of membranes (200 microliters) to 96 well V-bottom polypropylene plates containing 25 microliters of drug dilutions and 25 microliters of radioligand (1 nM final concentration ³H-N-methylhistamine). After a 1-hour incubation, assay samples are rapidly filtered through Whatman GF/B filters and rinsed with ice-cold 50 mM Tris buffer (pH 7.4) using a Skatron cell harvester. Radioactivity is quantified using a BetaPlate scintillation counter. The percent inhibition of specific binding can then be determined for each dose of the compound, and an IC50 or Ki value can be calculated from these results.

TABLE 1
Rat H3 Binding for selected compounds

| Example # | Rat H3 activity (Kⱼ, nM) |
|---|---|
| Int. 3 | 86 |
| 2 | 116 |
| 3 | 78 |
| 14 | 51 |
| 15 | 41 |
| 17 | 32 |
| 30 | 51 |
| 47 | 43 |
| 49 | 45 |
| 51 | 58 |
| 57 | 30 |
| 67 | 63 |
| 69 | 53 |
| 70 | 69 |

1. A compound of formula I 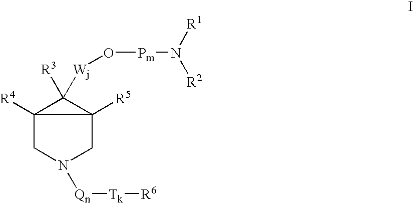
or the pharmaceutically acceptable salt(s) thereof, wherein:
P=methylene or a 3-8 member carbocyclic ring, optionally substituted by C₁-C₃ alkyl or fluorine;
T=methylene, optionally substituted by C₁-C₆ alkyl or cycloalkyl, OH, CN or phenyl (optionally substituted by Z as defined below);
Q=—(C═O)—, —SO₂—;
W═CR⁸R⁹;
j=0, 1 or 2;
k=0 to 6;
m=1 to 4;
n=0 or 1;
R¹ and R² are independently selected from the group that includes hydrogen, C₁-C₆ alkyl or C₃-C₇ cycloalkyl;
R¹ and R² together with the nitrogen to which they are attached form a 3-10 member cyclic or bicyclic ring, optionally with up to two additional heteroatoms selected from N, O, or S (e.g., azetidine, pyrrolidine, piperidine, azepine, piperazine, morpholine, thiomorpholine);
R³, R⁴ and R⁵ are independently selected from the group consisting of hydrogen; C₁-C₄ alkyl, optionally substituted with 1-4 halogens (especially fluorine) or OH;
C₃-C₇ cycloalkyl;
R⁶ is selected from the group that includes:
aryl, optionally substituted with Z;
heteroaryl, optionally substituted with Z;
R⁷ is selected from the list that includes:
hydrogen;
C₁-C₄ alkyl;
C₃-C₇ cycloalkyl;
R⁸ and R⁹ are independently selected from the group that includes:
hydrogen;
C₁-C₄ alkyl;
phenyl, optionally substituted with up to three of the atoms or functional groups defined for X below; or
R⁸ and R⁹ together with the carbon to which they are attached form a 3-7 member carbocyclic ring; and
X, Y and Z are independently selected from the group consisting of H, F, Cl, Br, I, CN, OH, NH₂, CF₃, C₂F₅, (C₁-C₆) alkyl, (C₁-C₆)-alkoxy or (C₁-C₆)alkyl-S(O)_{q}—, wherein q is 0, 1 or 2.

2. The compound of claim 1, wherein R¹ and R² together with the nitrogen to which they are attached form a piperidine ring.

3. The compound of claim 1, wherein R¹ and R² together with the nitrogen to which they are attached form a pyrrolidine ring.

4. The compound of claim 1, wherein R¹ and R² are each independently methyl.

5. The compound of claim 1, wherein R¹ and R² together with the nitrogen to which they are attached form a piperidine ring, R³, R⁴ and R⁵ are hydrogen, R⁶ is phenyl, k=0, m=3 and n=1.

6. The compound 6-hydroxymethyl-3-azabicyclo[3.1.0]hexane-3-carboxylic acid tert-butyl ester.

7. The compound 6-(3-piperidin-1-ylpropoxymethyl)-3-azabicyclo[3.1.0]hexane-3-carboxylic acid tert-butyl ester.

8. The compound 3-benzyl-6-(3-piperidin-1-ylpropoxymethyl)-3-azabicyclo[3.1.0]hexane.

9. The compound 6-(3-piperidin-1-ylpropoxymethyl)-3-azabicyclo[3.1.0]hexane.

10. The compounds of formula I in accordance with the present invention are the following:
3-benzyl-6-(3-piperidin-1-ylpropoxymethyl)-3-azabicyclo[3.1.0]hexane;
1-{4-[6-(3-piperidin-1-ylpropoxymethyl)-3-azabicyclo[3.1.0]hex-3-ylmethyl]-phenyl}-ethanone;
(1S,5R,6R)-3-naphthalen-2-ylmethyl-6-(3-piperidin-1-ylpropoxymethyl)-3-azabicyclo[3.1.0]hexane;
(1S,5R,6R)-6-(3-piperidin-1-ylpropoxymethyl)-3-pyridin-3-ylmethyl-3-azabicyclo[3.1.0]hexane;
(1S,5R,6R)-3-(4-methoxybenzyl)-6-(3-piperidin-1-ylpropoxymethyl)-3-azabicyclo[3.1.0]hexane;
(1S,5R,6R)-3-(4-methylbenzyl)-6-(3-piperidin-1-ylpropoxymethyl)-3-azabicyclo[3.1.0]hexane;
(1S,5R,6R)-3-(3-fluorobenzyl)-6-(3-piperidin-1-ylpropoxymethyl)-3-azabicyclo[3.1.0]hexane;
3-[(1S,5R,6R)-6-(3-piperidin-1-ylpropoxymethyl)-3-azabicyclo[3.1.0]hex-3-ylmethyl]-phenol;
4-[(1S,5R,6R)-6-(3-piperidin-1-ylpropoxymethyl)-3-azabicyclo[3.1.0]hex-3-ylmethyl]-phenol;
4-[(1S,5R,6R)-6-(3-piperidin-1-ylpropoxymethyl)-3-azabicyclo[3.1.0]hex-3-ylmethyl]-benzonitrile;
(1S,5R,6R)-3-(3-phenoxybenzyl)-6-(3-piperidin-1-ylpropoxymethyl)-3-azabicyclo[3.1.0]hexane;
(1S,5R,6R)-3-(3-benzyloxybenzyl)-6-(3-piperidin-1-ylpropoxymethyl)-3-azabicyclo[3.1.0]hexane;
(1S,5R,6R)-3-(4-butoxybenzyl)-6-(3-piperidin-1-ylpropoxymethyl)-3-azabicyclo[3.1.0]hexane;
(1S,5R,6R)-3-biphenyl-4-ylmethyl-6-(3-piperidin-1-ylpropoxymethyl)-3-azabicyclo[3.1.0]hexane;
(1S,5R,6R)-3-benzo[1,3]dioxol-5-ylmethyl-6-(3-piperidin-1-ylpropoxymethyl)-3-azabicyclo[3.1.0]hexane;
(1S,5R,6R)-6-(3-piperidin-1-ylpropoxymethyl)-3-(3-trifluoromethylbenzyl)-3-azabicyclo[3.1.0]hexane;
(1S,5R,6R)-3-(4-bromobenzyl)-6-(3-piperidin-1-ylpropoxymethyl)-3-azabicyclo[3.1.0]hexane;
(1S,5R,6R)-3-(4-isopropylbenzyl)-6-(3-piperidin-1-ylpropoxymethyl)-3-azabicyclo[3.1.0]hexane;
(1S,5R,6R)-3-(3-chlorobenzyl)-6-(3-piperidin-1-ylpropoxymethyl)-3-azabicyclo[3.1.0]hexane;
(1S,5R,6R)-3-(2,3-dihydro-benzo[1,4]dioxin-6-ylmethyl)-6-(3-piperidin-1-ylpropoxymethyl)-3-azabicyclo[3.1.0]hexane;
(1S,5R,6R)-3-(4-ethoxybenzyl)-6-(3-piperidin-1-ylpropoxymethyl)-3-azabicyclo[3.1.0]hexane;
(1S,5R,6R)-3-(4-tert-butylbenzyl)-6-(3-piperidin-1-ylpropoxymethyl)-3-azabicyclo[3.1.0]hexane;
3-[(1S,5R,6R)-6-(3-piperidin-1-ylpropoxymethyl)-3-azabicyclo[3.1.0]hex-3-ylmethyl]-benzonitrile;
(1S,5R,6R)-3-(3,5-dichlorobenzyl)-6-(3-piperidin-1-ylpropoxymethyl)-3-azabicyclo[3.1.0]hexane;
(1S,5R,6R)-3-benzo[1,3]dioxol-4-ylmethyl-6-(3-piperidin-1-ylpropoxymethyl)-3-azabicyclo[3.1.0]hexane;
(1S,5R,6R)-6-(3-piperidin-1-ylpropoxymethyl)-3-(4-trifluoromethylbenzyl)-3-azabicyclo[3.1.0]hexane;
(1S,5R,6R)-3-(4-phenoxybenzyl)-6-(3-piperidin-1-ylpropoxymethyl]-3-azabicyclo[3.1.0]hexane;
(1S,5R,6R)-3-(2,6-difluorobenzyl)-6-(3-piperidin-1-ylpropoxymethyl)-3-azabicyclo[3.1.0]hexane;
(1S,5R,6R)-3-(4-methylsulfanylbenzyl)-6-(3-piperidin-1-ylpropoxymethyl)-3-azabicyclo[3.1.0]hexane;
(1R,5S,6R)-3-(3,5-difluorobenzyl)-6-[(3-piperidin-1-ylpropyl)oxymethyl]-3-azabicyclo[3.1.0]hexane;
(1R,5S,6R)-6-(3-piperidin-1-ylpropoxymethyl)-3-(4-propoxybenzyl)-3-azabicyclo[3.1.0]hexane;
(1R,5S,6R)-3-(4-fluoro-3-trifluoromethylbenzyl)-6-[(3-piperidin-1-ylpropyl)oxymethyl]-3-azabicyclo[3.1.0]hexane;
(1R,5S,6R)-3-(4-tert-butoxybenzyl)-6-(3-piperidin-1-ylpropoxymethyl)-3-azabicyclo[3.1.0]hexane;
5-[(1R,5S,6R)-6-(3-piperidin-1-ylpropoxymethyl)-3-azabicyclo[3.1.0]hex-3-ylmethyl]-benzene-1,3-diol;
(1R,5S,6R)-6-(3-piperidin-1-ylpropoxymethyl)-3-(4-trifluoromethoxybenzyl)-3-azabicyclo[3.1.0]hexane;
(1R,5S,6R)-3-(3-ethoxy-4-methoxybenzyl)-6-[(3-piperidin-1-ylpropyl)oxymethyl]-3-azabicyclo[3.1.0]hexane;
(1R,5S,6R)-6-(3-piperidin-1-ylpropoxymethyl)-3-(4-trifluoromethylsulfanylbenzyl)-3-azabicyclo[3.1.0]hexane;
(1R,5S,6R)-3-(4-ethylbenzyl)-6-(3-piperidin-1-ylpropoxymethyl)-3-azabicyclo[3.1.0]hexane;
(1R,5S,6R)-3-(4-isopropoxybenzyl)-6-(3-piperidin-1-ylpropoxymethyl)-3-azabicyclo[3.1.0]hexane;
(1R,5S,6R)-3-(3,5-dimethylbenzyl)-6-[(3-piperidin-1-ylpropyl)oxymethyl]-3-azabicyclo[3.1.0]hexane;
(1R,5S,6R)-3-(2′-methylbiphenyl-4-ylmethyl)-6-(3-piperidin-1-ylpropoxymethyl)-3-azabicyclo[3.1.0]hexane;
2-{4-[(1R,5S,6R)-6-(3-piperidin-1-ylpropoxymethyl)-3-azabicyclo[3.1.0]hex-3-ylmethyl]-phenoxy}-ethanol;
(1R,5S,6R)-3-(4-isobutylbenzyl)-6-(3-piperidin-1-ylpropoxymethyl)-3-azabicyclo[3.1.0]hexane;
(1R,5S,6R)-3-[4-(4-fluorophenoxy)-benzyl]-6-(3-piperidin-1-ylpropoxymethyl)-3-azabicyclo[3.1.0]hexane;
(1R,5S,6R)-3-(2,2-dimethylchroman-6-ylmethyl)-6-[(3-piperidin-1-ylpropyl)oxymethyl]-3-azabicyclo[3.1.0]hexane;
N-{4-[(1R,5S,6R)-6-(3-piperidin-1-ylpropoxymethyl)-3-azabicyclo[3.1.0]hex-3-ylmethyl]-phenyl}-acetamide;
6-[(1R,5S,6R)-6-(3-piperidin-1-ylpropoxymethyl)-3-azabicyclo[3.1.0]hex-3-ylmethyl]-quinoxaline;
1-{4-[(1R,5S,6R)-6-(3-piperidin-1-ylpropoxymethyl)-3-azabicyclo[3.1.0]hex-3-ylmethyl]-phenyl}-imidazolidin-2-one;
(1R,5S,6R)-3-(4-benzyloxybenzyl)-6-(3-piperidin-1-ylpropoxymethyl)-3-azabicyclo[3.1.0]hexane;
(1R,5S,6R)-3-(4-pentyloxybenzyl)-6-(3-piperidin-1-ylpropoxymethyl)-3-azabicyclo[3.1.0]hexane;
(1R,5S,6R)-6-(3-piperidin-1-ylpropoxymethyl)-3-[4-(1H-tetrazol-5-yl)-benzyl]-3-azabicyclo[3.1.0]hexane;
3-(methyl-{4-[(1R,5S,6R)-6-(3-piperidin-1-ylpropoxymethyl)-3-azabicyclo[3.1.0]hex-3-ylmethyl]-phenyl}-amino)-propionitrile;
5-[(1R,5S,6R)-6-(3-piperidin-1-ylpropoxymethyl)-3-azabicyclo[3.1.0]hex-3-ylmethyl]-1H-indole;
(1R,5S,6R)-3-(4′-methoxybiphenyl-4-ylmethyl)-6-(3-piperidin-1-ylpropoxymethyl)-3-azabicyclo[3.1.0]hexane;
4-methyl-7-[(1R,5S,6R)-6-(3-piperidin-1-ylpropoxymethyl)-3-azabicyclo[3.1.0]hex-3-ylmethyl]-3,4-dihydro-2H-benzo[1,4]oxazine;
(1R,5S,6R)-3-[3-(cyclopent-3-ethoxy)-benzyl]-6-(3-piperidin-1-ylpropoxymethyl)-3-azabicyclo[3.1.0]hexane;
(1R,5S,6R)-6-(3-piperidin-1-ylpropoxymethyl)-3-[3-(1,1,2,2-tetrafluoroethoxy)-benzyl]-3-azabicyclo[3.1.0]hexane;
(1R,5S,6R)-3-(4-morpholin-4-ylbenzyl)-6-(3-piperidin-1-ylpropoxymethyl)-3-azabicyclo[3.1.0]hexane;
(1R,5S,6R)-3-[4-(cyclopent-3-enyloxy)-benzyl]-6-(3-piperidin-1-ylpropoxymethyl)-3-azabicyclo[3.1.0]hexane;
(1R,5S,6R)-6-(3-piperidin-1-ylpropoxymethyl)-3-(4-[1,2,4]triazol-1-ylbenzyl)-3-azabicyclo[3.1.0]hexane;
2-{4-[(1R,5S,6R)-6-(3-piperidin-1-ylpropoxymethyl)-3-azabicyclo[3.1.0]hex-3-ylmethyl]-phenoxy}-acetamide;
(1R,5S,6R)-6-(3-piperidin-1-ylpropoxymethyl)-3-(4-pyrimidin-5-ylbenzyl)-3-azabicyclo[3.1.0]hexane;
(1R,5S,6R)-3-(3-methoxybenzyl)-6-(3-piperidin-1-ylpropoxymethyl)-3-azabicyclo[3.1.0]hexane;
(1R,5S,6R)-6-(3-piperidin-1-ylpropoxymethyl)-3-(4-pyridin-3-ylbenzyl)-3-azabicyclo[3.1.0]hexane;
(1R,5S,6R)-3-(2,2-difluorobenzo[1,3]dioxol-5-ylmethyl)-6-[(3-piperidin-1-ylpropyl)oxymethyl]-3-azabicyclo[3.1.0]hexane;
(1R,5S,6R)-6-(3-piperidin-1-ylpropoxymethyl)-3-[4-(1,1,2,2-tetrafluoroethoxy)-benzyl]-3-azabicyclo[3.1.0]hexane;
(1R,5S,6R)-3-(4-isobutoxybenzyl)-6-(3-piperidin-1-ylpropoxymethyl)-3-azabicyclo[3.1.0]hexane;
(1R,5S,6R)-6-(3-piperidin-1-ylpropoxymethyl)-3-(4-pyrazol-1-ylbenzyl)-3-azabicyclo[3.1.0]hexane;
(1R,5S,6R)-3-(2-chlorobenzyl)-6-(3-piperidin-1-ylpropoxymethyl)-3-azabicyclo[3.1.0]hexane;
(1R,5S,6R)-3-(2,2-difluorobenzo[1,3]dioxol-4-ylmethyl)-6-[(3-piperidin-1-ylpropyl)oxymethyl]-3-azabicyclo[3.1.0]hexane;
(1R,5S,6R)-3-(2,3-dihydrobenzofuran-5-ylmethyl)-6-(3-piperidin-1-ylpropoxymethyl)-3-azabicyclo[3.1.0]hexane;
3-[(1S,5R,6R)-6-(3-piperidin-1-ylpropoxymethyl)-3-azabicyclo[3.1.0]hex-3-yl]-benzo[d]isoxazole;(1R,5S,6R)-3-phenethyl-6-(3-piperidin-1-ylpropoxymethyl)-3-azabicyclo[3.1.0]-hexane; and
(1R,5S,6R)-3-(4-chlorobenzyl)-6-(3-piperidin-1-ylpropoxymethyl)-3-azabicyclo[3.1.0]hexane.

11. The compound of formula I, wherein the compound is selected from the group consisting of:
3-(3,4-dichlorobenzyl)-6-(3-piperidin-1-ylpropoxymethyl)-3-azabicyclo[3.1.0]hexane;
3-(4-chlorobenzyl)-6-(3-morpholin-4-ylpropoxymethyl)-3-azabicyclo[3.1.0]hexane;
3-(4-methoxybenzyl)-6-(3-thiomorpholin-4-ylpropoxymethyl)-3-azabicyclo[3.1.0]-hexane;
N-isopropyl-N-methyl-[3-(3-pyridin-2-ylmethyl-3-azabicyclo[3.1.0]hex-6-ylmethoxy)-propyl]-amine;
[2-(3-pyrimidin-2-ylmethyl-3-azabicyclo[3.1.0]hex-6-ylmethoxy)-ethyl]-dicyclopropyl amine;
{6-[2-(2,4-dimethylazetidin-1-yl)-ethoxymethyl]-3-azabicyclo[3.1.0]hex-3-yl}-pyridin-4-ylmethanone;
6-(2-pyrrolidin-1-ylethoxymethyl)-3-azabicyclo[3.1.0]hexane-3-carboxylic acid methyl-(3-trifluoromethylphenyl)-amide;
1,5-dimethyl-3-phenethyl-6-(2-pyrrolidin-1-ylethoxymethyl)-3-azabicyclo[3.1.0]hexane;
[2-(3-benzenesulfonyl-3-azabicyclo[3.1.0]hex-6-ylmethoxy)-ethyl]-diethylamine;
{2-[3-(1H-indole-6-sulfonyl)-3-azabicyclo[3.1.0]hex-6-ylmethoxy]-ethyl}-dimethylamine;
[6-(2-dimethylamino-ethoxymethyl)-6-methyl-3-azabicyclo[3.1.0]hex-3-yl]-phenyl-methanone;
6-(2-pyrrolidin-1-ylethoxymethyl)-3-azabicyclo[3.1.0]hexane-3-carboxylic acid phenyl-amide;
6-(3-pyrrolidin-1-ylpropoxymethyl)-3-azabicyclo[3.1.0]hexane-3-carboxylic acid p-tolylamide;
1-{4-[2-(3-benzyl-3-azabicyclo[3.1.0]hex-6-ylmethoxy)-ethyl]-piperazin-1-yl}-ethanone;
3-benzyl-6-[2-(4-methanesulfinyl-piperazin-1-yl)-ethoxymethyl]-3-azabicyclo[3.1.0]-hexane;
3-benzyl-6-{2-[4-(propane-2-sulfonyl)-piperazin-1-yl]-ethoxymethyl}3-azabicyclo[3.1.0]hexane;
3-benzyl-6-{2-[4-(4-chlorobenzenesulfonyl)-piperazin-1-yl]-ethoxymethyl}-3-azabicyclo[3.1.0]hexane;
3-benzyl-6-{2-[4-(3-fluorophenyl)-piperazin-1-yl]-ethoxymethyl}-3-azabicyclo[3.1.0]-hexane;
3-benzyl-6-[2-(4-pyridin-2-ylpiperazin-1-yl)-ethoxymethyl]-3-azabicyclo[3.1.0]hexane;
3-(2-methylbenzyl)-6-[2-(4-pyrimidin-2-ylpiperazin-1-yl)-ethoxymethyl]-3-azabicyclo[3.1.0]hexane;
6-[2-(2,5-dimethylpyrrolidin-1-yl)-ethoxymethyl]-3-(2-methoxybenzyl)-3-azabicyclo[3.1.0]hexane;
{2-[3-(1-phenylethyl)-3-azabicyclo[3.1.0]hex-6-ylmethoxy]-ethyl}-dimethylamine;
3-benzyl-6-[3-(3,5-dimethylmorpholin-4-yl)-propoxymethyl]-3-azabicyclo[3.1.0]hexane;
3-benzyl-6-(1-methyl-2-pyrrolidin-1-ylethoxymethyl)-3-azabicyclo[3.1.0]hexane;
[6-(2-pyrrolidin-1-ylpropoxymethyl)-3-azabicyclo[3.1.0]hex-3-yl]-phenyl-methanone;
[6-(2-dimethylamino-ethoxymethyl)-3-azabicyclo[3.1.0]hex-3-yl]-(6-fluoronaphthalen-2-yl)-methanone;
[6-(2-dimethylamino-propoxymethyl)-3-azabicyclo[3.1.0]hex-3-yl]-naphthalen-2-yl-methanone;
[6-(2-dimethylamino-propoxymethyl)-3-azabicyclo[3.1.0]hex-3-yl]-quinolin-3-yl-methanone;
[6-(2-pyrrolidin-1-ylpropoxymethyl)-3-azabicyclo[3.1.0]hex-3-yl]-quinolin-3-yl-methanone;
3-benzyl-6-(4-piperidin-1-yl butoxymethyl)-3-azabicyclo[3.1.0]hexane;
(4-methoxyphenyl)-[6-(4-piperidin-1-ylbutoxymethyl)-3-azabicyclo[3.1.0]hex-3-yl]-methanone;
3-(3-chlorobenzenesulfonyl)-6-(4-pyrrolidin-1-ylbutoxymethyl)-3-azabicyclo[3.1.0]-hexane;
3-benzenesulfonyl-1,5-dimethyl-6-(4-pyrrolidin-1-ylbutoxymethyl)-3-azabicyclo[3.1.0]-hexane;
3-benzyl-6-(4-piperidin-1-ylcyclohexyloxymethyl)-3-azabicyclo[3.1.0]hexane;
[3-(3-benzyl-3-azabicyclo[3.1.0]hex-6-ylmethoxy)-cyclopentyl]-dimethylamine;
3-benzyl-6-(3-morpholin-4-ylcyclobutoxymethyl)-3-azabicyclo[3.1.0]hexane;
3-(4-chlorobenzyl)-6-(2-pyrrolidin-1-ylcyclopropoxymethyl)-3-azabicyclo[3.1.0]hexane;
3-benzyl-6-(3-pyrrolidin-1-ylbicyclo[3.2.1]oct-8-yloxymethyl)-3-azabicyclo[3.1.0]-hexane; and
[5-(3-benzyl-3-azabicyclo[3.1.0]hex-6-ylmethoxy)-octahydro-pentalen-2-yl]-dimethylamine.

12. A pharmaceutical composition for treating a disorder or condition that may be treated by antagonizing histamine-3 receptors, the composition comprising a compound of formula I as described in claim 1, and optionally a pharmaceutically acceptable carrier.

13. A method of treatment of a disorder or condition that may be treated by antagonizing histamine-3 receptors, the method comprising administering to a mammal in need of such treatment a compound of formula I as described in claim 1.

14. A pharmaceutical composition comprising a compound of formula I as described in claim 1, and optionally a pharmaceutically acceptable carrier.

15. A method of treatment of a disorder or condition selected from the group consisting of depression, mood disorders, schizophrenia, anxiety disorders, Alzheimer's disease, attention-deficit hyperactivity disorder (ADHD), psychotic disorders, sleep disorders, obesity, dizziness, epilepsy, motion sickness, respiratory diseases, allergy, allergy-induced airway responses, allergic rhinitis, nasal congestion, allergic congestion, congestion, hypotension, cardiovascular disease, diseases of the GI tract, hyper and hypo motility and acidic secretion of the gastro-intestinal tract, the method comprising administering to a mammal in need of such treatment a compound of formula I as described in claim 1.

16. The method of claim 15, wherein the disorder or condition is selected from the group consisting of anxiety disorders, attention-deficit hyperactivity disorder, respiratory diseases, and obesity.

17. The method of claim 15, wherein the disorder or condition is a respiratory disease selected from the group consisting of adult respiratory distress syndrome, acute respiratory distress syndrome, bronchitis, chronic bronchitis, chronic obstructive pulmonary disease, cystic fibrosis, asthma, emphysema, rhinitis and chronic sinusitis.

18. A pharmaceutical composition for treating allergic rhinitis, nasal congestion or allergic congestion comprising:
a) an H3 receptor antagonist compound of formula I; or a pharmaceutically acceptable salt thereof;
b) an H1 receptor antagonist such as cetirizine; or a pharmaceutically acceptable salt thereof; and
c) a pharmaceutically acceptable carrier;
wherein the active ingredients (a) and (b) above are present in amounts that render the composition effective in treating allergy rhinitis, nasal congestion or allergic congestion

19. A pharmaceutical composition for treating depression and mood disorder comprising:
a) an H3 receptor antagonist or a pharmaceutically acceptable salt thereof;
b) a neurotransmitter uptake blocker or
c) a pharmaceutically acceptable salt thereof;
wherein the active ingredients (a) and (b) above are present in amounts that render the composition effective in treating depression and mood disorder.

20. The composition according to claim 19 wherein the H3 receptor antagonist and the neurotransmitter blocker are given simultaneously.

21. The composition according to claim 18 wherein the H3 receptor antagonist and the H1 receptor antagonist are given simultaneously.

22. The pharmaceutical composition of claim 19 wherein the neurotransmitter uptake blocker are selected from SSRI; sertraline, fluoxetine and paroxetine.
